(19) United States
(12) Patent Application Publication
(10) Pub. No.: US 2025/0129332 A1
(43) Pub. Date: Apr. 24, 2025

(54) METHODS OF SORTING CELLS FOR PHOTORECEPTOR TRANSPLANTATION TREATMENT

(71) Applicant: THE JOHNS HOPKINS UNIVERSITY, Baltimore, MD (US)

(72) Inventors: Mandeep Singh, Baltimore, MD (US); Seth Blackshaw, Baltimore, MD (US); Robert Johnston, Baltimore, MD (US); Ying Liu, Baltimore, MD (US); Clayton Santiago, Baltimore, MD (US)

(73) Assignee: THE JOHNS HOPKINS UNIVERSITY, Baltimore, MD (US)

(21) Appl. No.: 18/936,315

(22) Filed: Nov. 4, 2024

Related U.S. Application Data
(63) Continuation of application No. PCT/US2023/020912, filed on May 4, 2023.
(60) Provisional application No. 63/338,318, filed on May 4, 2022.

(51) Int. Cl. C12N 5/079; A61K 35/30; A61P 27/02; C12N 5/00; C12N 5/071; C12N 9/64; C12N 9/76

(52) U.S. Cl. CPC C12N 5/0621; A61K 35/30; A61P 27/02; C12N 5/0081; C12N 5/0697; C12N 9/6427; C12N 9/6472; C12Y 304/21004; C12Y 304/22002; C12N 2509/00

(57) ABSTRACT

The present disclosure provides methods for sorting retinal cells for use in cellular component transfer therapy, sorted populations of retinal cells generated by such methods, and compositions comprising such sorted populations of retinal cells. The present disclosure also provides uses of sorted populations of retinal s and compositions comprising thereof for preventing and/or treating inherited retinal degenerative diseases.

### CROSS-REFERENCE TO RELATED APPLICATIONS

This application is a continuation of International Application No. PCT/US2023/020912 filed May 4, 2023, which claims priority to U.S. Provisional Application No. 63/338,318 filed May 4, 2022, the contents of both of which are incorporated by reference in their entireties, and to which priority is claimed.

### STATEMENT REGARDING FEDERALLY SPONSORED RESEARCH

This invention was made with government support under grants EY029157, EY007143, EY030872, EY001765 and EY033103 awarded by the National Institutes of Health. The government has certain rights in the invention.

### SEQUENCE LISTING

A Sequence Listing conforming to the rules of WIPO Standard ST.26 is hereby incorporated by reference. Said Sequence Listing has been filed as an electronic document via Patent Center in ASCII format encoded as XML. The electronic document, created on Oct. 31, 2024, is entitled “088933.0139.xml”, and is 8,895 bytes in size.

### INTRODUCTION

The present disclosure provides methods for sorting retinal cells for use in cellular component transfer therapy, sorted populations of retinal cells generated by such methods, and compositions comprising such sorted populations of retinal cells. The present disclosure also provides uses of the sorted populations of retinal cells and compositions comprising thereof for preventing and/or treating inherited or acquired retinal degenerative diseases.

### BACKGROUND

Photoreceptor cell transplantation is currently being developed as a treatment for blindness resulting from a variety of inherited or acquired retinal degenerative diseases. In one approach, subretinal transplantation of retinal cells results in the therapeutic transfer of cytoplasm and other cellular components including but not limited to macromolecules, mitochondria, endoplasmic reticulum, peroxisomes and lysosomes, nucleic acids, cilium components, and membrane components from donor to host cells. In contrast to any expectation that donor cells will lodge as independently functionally photoreceptors with new synaptic connections with downstream neurons, cellular component transfer therapy (“CCTT”) acts by repairing and/or promoting the health, longevity, and/or functionality of the dysfunctional photoreceptor cells already present in the recipient's retina. With the appropriate donor cell preparation, this concept of intercellular components transfer is predicted to be therapeutically important in other neuronal targets such as dopaminergic neurons (Parkinson disease), entorhinal cortex and hippocampus (Alzheimer disease), and sensory hair cells of the inner ear (sensorineural deafness). Despite recent advances in cell culture strategies allowing for the production retinal organoids as a source of donor photoreceptor cells, there remains a need for improved methods for generating sorted populations of retinal cells suitable for efficiently and effectively treating inherited or acquired retinal degenerative diseases via CCTT. In particular, the goals of cell sorting include depletion of potentially deleterious or inactive cells and enrichment of therapeutically active cells.

### SUMMARY OF THE INVENTION

The present disclosure provides methods for generating sorted populations of retinal cells for use in CCTT, sorted populations of retinal cells generated by such methods, and compositions comprising such sorted populations of retinal cells. The present disclosure also provides uses of the sorted populations of retinal cells and compositions comprising thereof for preventing and/or treating inherited or acquired retinal degenerative diseases.

In certain embodiments, the present disclosure is directed to methods, e.g. in vitro methods, to produce sorted population of retinal cells. In certain embodiments, such methods comprise: generating a three-dimensional retinal organoid; dissociating the three-dimensional retinal organoid; and positively sorting retinal cells based on one or more marker of photoreceptor cell identity and/or negatively sorting retinal cells based on one or more marker of non-photoreceptor cell identity to produce the sorted population of retinal cells. In certain embodiments, the marker of photoreceptor cell identity is CD73. In certain embodiments, the marker of non-photoceptor cell identity is one or more of CD24, CD302,CD9, and CD99. In certain embodiments, the marker of non-photoreceptor cell identity is one or more of ITM2B; CD63; ENO1; CALR; CANX; CLU; SLC3A2; BSG; GPM6B; ITGB1; PTTG1IP; TIMP1; PMEPA1; SSR2; DKK3; LRP1; ATRAID; HLA-A; HLA-C; EMP3; TMED9; GOLIM4; LTBP3; GALNT1; CD151; PLD3; CALU; LSAMP; CD59; SLC2A1; LAMP2; HLA-B; COL11A1; DPP7; DCBLD2; CD164; SLC1A3; F3; CTSD; FLNA; SLC39A10; FN1; TMEM106C; TMEM179B; ATP1B3; HLA-E; TMEM132A; FLT1; FGFR1; CAPNS1; FAT1; ANGPTL1; LRP10; CRELD2; SPPL2A; TSPAN4; PRSS35; ECE1; SYPL1; SORCS2; COL2A1; DNER; COL6A1; CD44; GPC1; PCDH9; CRIM1; CHL1; TTYH3; IKBIP; NECTIN2; FBLN2; CCDC80; DAG1; PBXIP1; PRSS23; ACAA1; NTRK2; FSTL1; BCHE; TNFRSF1A; LGALS3BP; ITGB8; CP; ADGRG1; VCAN; OLFM1; NRP1; SCARB1; TSPAN6; PCDH17; PLTP; NECTIN3; PTPRD; CADM4; UNC5B; CSPG5; AXL; PLXNB2; PLPP3; NOTCH2; SLITRK2; AEBP1; ANGPTL4; COLEC12; VAMP5; NLGN4X; FGFRL1; EFNB2; COL5A1; LAMB2; LAMC1; IGFBP3; FNDC5; FCGRT; ADRA2C; SERPING1; EPHB2; CDH11; COL1A2; CNTFR; AGRN; ROBO1; LOX; MRC2; COL6A2; SLC6A11; DSC2; IGSF8; EMP1; ABI3BP; TTYH2; NOTCH1; ANO6; A2M; SORCS1; EFNA1; PTPRG; TF; EMP2; CEMIP2; SERPINE2; CDON; EGFR; PCDH7; MAN2A1; IL1R1; COL1A1; SEMA5A; ANTXR1; S1PR3; ITPRIP; MXRA8; PRELP; AQP1; CSF1; BCAN; ADGRA2; CA12; FAT3; HEPACAM; FGFR3; TRIL; HSD17B2; and HP. In certain embodiments, the marker of non-photoreceptor cell identity is one or more of DKK3; LRP1; CLU; PMEPA1; ITGB1; and PTTG1IP.

In certain embodiments, the one or more marker of non-photoreceptor cell identity is one or more astrocyte marker. In certain embodiments, the one or more astrocyte marker is selected from: ITM2B; CD63; ENO1; CALR; CANX; CLU; SLC3A2; BSG; GPM6B; ITGB1; PTTG1IP; TIMP1; PMEPA1; SSR2; DKK3; LRP1; ATRAID; HLA-A; HLA-C; EMP3; TMED9; GOLIM4; LTBP3; GALNT1; CD151; PLD3; CALU; LSAMP; CD59; SLC2A1; LAMP2; HLA-B; COL11A1; DPP7; DCBLD2; CD164; SLC1A3; F3; CTSD; FLNA; SLC39A10; FN1; TMEM106C; TMEM179B; ATP1B3; HLA-E; TMEM132A; FLT1; FGFR1; CAPNS1; FAT1; ANGPTL1; LRP10; CRELD2; SPPL2A; TSPAN4; PRSS35; ECE1; SYPL1; SORCS2; COL2A1; DNER; COL6A1; CD44; GPC1; PCDH9; CRIM1; CHL1; TTYH3; IKBIP; NECTIN2; FBLN2; CCDC80; DAG1; PBXIP1; PRSS23; ACAA1; NTRK2; FSTL1; BCHE; TNFRSF1A; LGALS3BP; ITGB8; CP; ADGRG1; VCAN; OLFM1; NRP1; SCARB1; TSPAN6; PCDH17; PLTP; NECTIN3; PTPRD; CADM4; UNC5B; CSPG5; AXL; PLXNB2; PLPP3; NOTCH2; SLITRK2; AEBP1; ANGPTL4; COLEC12; VAMP5; NLGN4X; FGFRL1; EFNB2; COL5A1; LAMB2; LAMC1; IGFBP3; FNDC5; FCGRT; ADRA2C; SERPING1; EPHB2; CDH11; COL1A2; CNTFR; AGRN; ROBO1; LOX; MRC2; COL6A2; SLC6A11; DSC2; IGSF8; EMP1; ABI3BP; TTYH2; NOTCH1; ANO6; A2M; SORCS1; EFNA1; PTPRG; TF; EMP2; CEMIP2; SERPINE2; CDON; EGFR; PCDH7; MAN2A1; IL1R1; COL1A1; SEMA5A; ANTXR1; SIPR3; ITPRIP; MXRA8; PRELP; AQP1; CSF1; BCAN; ADGRA2; CA12; FAT3; HEPACAM; FGFR3; TRIL; HSD17B2; and HP. In certain embodiments, the one or more marker of non-photoreceptor cell identity is one or more astrocyte marker selected from ADGRL4; SERPINE2; BCHE; ABI3BP; NRP1; FSTL1; FAT1; NTRK2; FBLN2; PRSS35; SLC1A3; FCGRT; LAMC1; TF; SORCS2; DKK3; LRP1; PTPRD; ANGPTL1; LTBP3; CLU; CNTNAP2; CD151; PCDH9; CRIM1; CSPG5; and PMEPA1.

In certain embodiments, the one or more marker of non-photoreceptor cell identity is one or more brain and spinal cord-like (BSL) cell marker. In certain embodiments, the one or more marker of non-photoreceptor cell identity is one or more BSL cell marker selected from CLU; ITM2B; PTPRZ1; GPM6B; ATP1B2; CD63; BCAN; SLC1A3; SERPINE2; LRP1; PTPRA; ADGRG1; ENO1; CANX; SLC3A2; DNER; PTTG1IP; CALR; PCDH9; CCDC80; LSAMP; HEPACAM; F3; PLPP3; APLP2; FBLN2; TIMP1; SLC6A11; CSPG5; JAM2; FGFR3; DKK3; GOLIM4; NCAM1; CHL1; NRCAM; HLA-A; TMEM132A; PMEPA1; ITGAV; SSR2; ACAA1; BCHE; CD59; FAT3; PCDH17; ST3GAL5; PBXIP1; LAMP1; ITGB1; HP; ITGB8; SGCB; LAMP2; CLDND1; TMEM106B; PTCH1; PLTP; RNF13; HLA-C; PTPRD; TMEM30A; TRIL; RAB5C; TTYH3; DAG1; CADM4; UBA1; SLC6A9; LRRC8A; ATP1B3; SPPL2A; NTRK2; RNF130; LIFR; EMP3; PCDH7; NTRK3; COL6A1; IL17D; LRP10; ADAM19; SGCE; FAT1; SLC44A1; LTBP3; SLC39A10; ABCA1; SYPL1; SLITRK2; GNPTG; CD302; MRC2; LRP4; CALU; CD151; SORL1; TSPAN6; LRRN1; TENM2; CAPNS1; NLGN1; SLC15A2; NLGN4X; EGFR; ADORA1; SLC9A7; SIRPA; EFCAB14; ANGPTL1; FGFR1; VAMP5; CLDN12; LAMB2; GPR155; FGFR2; SLC44A2; LRP1B; PTGFRN; FNDC5; NOTCH1; DPY19L4; SIPR1; CD44; TNFRSF1A; FAM234A; CDH4; HLA-E; COL11A1; NCAM2; AQP1; CPQ; EMP1; FCGRT; GPC5; ROBO1; VCAN; LGALS3BP; LDLR; LRRC4B; NOTCH2; ALCAM; RYR3; SLC9A9; TMEM94; VCAM1; IGSF1; PCDHA10; CDH10; CACHD1; P2RX7; AEBP1; PLXNB1; AXL; ALPL; ST3GAL4; SERPINI1; ITGB5; CD58; FGFRL1; PLPP1; TTYH2; IL17RB; FAM171A1; IL17RD; ANO6; ADAM22; PTPRG; ANTXR1; ZDHHC23; AGRN; COL14A1; POSTN; CNTFR; SEMA5A; FLNA; EMP2; TFPI; ITGA7; MXRA8; TENM4; FSTL1; CD82; NRP2; GPC4; ARSF; LAMC1; KIT; SEMA4A; LTBP1; and CSF1. In certain embodiments, the one or more marker of non-photoreceptor cell identity is one or more BSL marker selected from HEPACAM; FGFR3; SERPINE2; BCAN; CCDC80; PLPP3; CHL1; ADGRG1; SLC6A11; LSAMP; FBLN2; F3; SLC1A3; DKK3; LRP1; DNER; CLU; PCDH9; and CSPG5.

In certain embodiments, the three-dimensional retinal organoid of the methods disclosed herein is enzymatically dissociated. In certain embodiments, the enzyme is papain and/or trypsin. In certain embodiments, the retinal cells are contacted with a composition to ensure that the cells remain in a dissociated cell suspension. In certain embodiments, the composition that ensures that the cells remain in a dissociated cell suspension is an enzyme. In certain embodiments, the enzyme is DNAse.

In certain embodiments of the methods of the present disclosure, the three-dimensional retinal organoid reaches between about DD 45 and DD 300 prior to being dissociated. In certain embodiments, the three-dimensional retinal organoid reaches about DD 90 to about DD 140 prior to being dissociated.

In certain embodiments of the methods of the present disclosure, the retinal cell population consists of at least about 70% single cells. In certain embodiments, the retinal cell population consists of at least about 80% single cells. In certain embodiments, the retinal cell population consists of at least about 90% single cells. In certain embodiments, the retinal cell population comprises about 55% to about 85% rod photoreceptor cells.

In certain embodiments of the methods of the present disclosure, the stem cells are selected from human, nonhuman primate or rodent nonembryonic stem cells; human, nonhuman primate or rodent embryonic stem cells; human, nonhuman primate or rodent induced pluripotent stem cells; and human, nonhuman primate or rodent recombinant pluripotent cells. In certain embodiments, the stem cells are human stem cells. In certain embodiments, the stem cells are pluripotent or multipotent stem cells. In certain embodiments, the stem cells are pluripotent stem cells. In certain embodiments, the pluripotent stem cells are selected from embryonic stem cells, induced pluripotent stem cells, and combinations thereof.

In certain embodiments, the present disclosure is directed to a sorted population of in vitro differentiated retinal cells, wherein said in vitro differentiated and sorted retinal cells are obtained by a method as described herein.

In certain embodiments, the present disclosure is directed to a composition comprising the in vitro differentiated and sorted retinal cells, wherein said in vitro differentiated sorted retinal cells are obtained by a method as described herein. In certain embodiments, the composition is a pharmaceutical composition comprising a sorted population of retinal cells and a pharmaceutically acceptable carrier.

In certain embodiments, the present disclosure is directed to methods of preventing and/or treating an inherited or acquired retinal degenerative disease in a subject, comprising administering to the subject an effective amount of one of the following: (a) a sorted population of retinal cells as described herein; or (b) a composition comprising a sorted population of retinal cells as described herein. In certain embodiments, the inherited retinal degenerative disease is selected from retinitis pigmentosa, choroideremia, Stargardt disease, cone-rod dystrophy, and Leber Congenital Amaurosis. In certain embodiments, the acquired retinal degenerative disease is age-related macular degeneration.

### BRIEF DESCRIPTION OF THE DRAWINGS

FIGS. 1A-1G show that human donor cells can exhibit short or long-range migration from the subretinal space of recipient mice. FIG. 1A depicts immunohistochemical (IHC) staining of human nucleus specific antibodies (HNA) showing migratory (arrows) and nonmigratory (empty arrow) cells of donor human retinal organoids in the recipient retina. FIGS. 1B and 1C shows the migratory cells were overlying the graft (radial migration) and beyond the graft edge (tangential migration). FIG. 1D shows a non-transplanted mouse eye negatively staining for HNA and CRX:tdTomato. FIG. 1E depicts the relative abundance of migratory human cells in different recipient retinal laminae (RGC, IPL, INL, RPE/C) (n=17 sections from five transplanted eyes). FIG. 1F shows quantification of the distance of the migratory human cell nuclei from the graft edge in different retinal laminae (RGC, IPL, INL, RPE/C) (n=21 sections from five transplanted eyes). FIG. 1G shows that migratory Ku80⁺ human cells found in the regions flanking the optic nerve (peripapillary migration). White arrows showed representative migratory human cells. Abbreviations: RGC: retinal ganglion cell; IPL: inner plexiform layer; INL: inner nuclear layer; RPE/C: retinal pigment epithelium and choroid. Yellow lines in FIGS. 1B, 1C, 1D, and 1G: the boundaries of the recipient retina; Green lines in FIG. 1G: optic nerve.

FIGS. 2A-2E show single-cell RNA sequencing analysis (scRNA-seq) revealing the retinal-derived and non-retinal-derived cell fates of transplanted and cultured donor retinal organoid cells. FIG. 2A depicts schematic showing the in vivo and in vitro control conditions of donor cells analyzed by scRNA-seq. CRX:tdTomato⁺ hESC derived-retinal organoids (aged 134 days) were transplanted into Rd1/NS mice or maintained in culture. Four and a half months later, single cell suspensions of transplanted and cultured retinal organoids (age-matched) were collected by papain dissociation and analyzed by Chromium scRNA seq. FIGS. 2B and 2C show that scRNA seq identified nine distinct cell clusters from the pool of transplanted and cultured retinal organoid cells (n=5,831 cells). FIG. 2D shows dot plots of marker gene expression in the identified cell clusters. The color scale corresponds to the average gene expression and the dot size corresponds to the percent of positively expressing cells in each cluster. FIG. 2E shows the relative abundance of cells of each type in transplanted and cultured retinal organoids.

FIGS. 3A-3G show that donor retinal astrocytes and brain/spinal cord-like (BSL) cells show long-range migratory capacity. FIG. 3A shows that scRNA seq analysis showed the highest migration score in the astrocytes and BSL cells among the cell types identified in transplanted retinal organoids. FIG. 3B shows that RNA scope staining showed migratory cells expressed markers (red) of astrocytes (PAX2, HES6) and BSL cells (ASCLI, HOXC8, NKX2-2, ARX). IHC counter-staining of human nuclear antibody Ku80 (white) was used to detected transplanted human cells. FIG. 3C shows relative abundance of migratory astrocytes and BSL cells in different recipients' retinal laminae. FIG. 3D shows quantification of migratory astrocytes and BSL cells in migratory human cells (n=3-4 eyes). FIGS. 3E and 3F show RNA scope staining (FIG. 1E) and quantification (FIG. 1F) of non-migratory astrocytes (PAX2) and BSL cells (ASCLI, HOXC8, NKX2-2, ARX) that remained in the subretinal space. IHC for Ku80 (white) facilitated detection of transplanted human cells (n=6-9 regions of interest from 4 transplanted eyes). FIG. 3G shows that migratory cells negatively express markers (green) of RGC (RBPMS, NeuN), amacrine cells (NeuN), horizontal cells (calbindin), rod bipolar cells (PKCα), cone bipolar cells (SCGN), microglia (IBA1), and macrophage (CD68). DAPI staining (blue) was performed to identify the nuclei of recipient retinal laminae.

FIGS. 4A-4E show that actively proliferating cells are rare among migratory and non-migratory donor cells. FIG. 4A shows that Ki-67⁺ proliferating cells were rare in cultured organoids (n=4 in one batch) and significantly less in transplanted organoids (n=3 eyes). FIG. 4B shows rare Ki-67⁺ cells detected among non-migratory and migratory cells in transplanted retinal organoids (n=5 eyes). FIG. 4C shows relative abundance of migratory Ki-67⁺ cells in different retinal laminae (RGC, IPL, INL, RPE/C). FIG. 4D shows that ScRNA-seq analysis showed the highest proliferation score in Müller glia, RPCs, astrocytes, and BSL cells among the identified cell types in transplanted retinal organoids (n=3 eyes). FIG. 4E shows that RNA scope and IHC show very few Ki67⁺ cells in migratory astrocytes (PAX2⁺) and BSL cells (ASCL1⁺), and non-migratory retinal progenitor cells (VSX2⁺) and BSL cells (HOXC8⁺) (n=4 eyes).

FIGS. 5A-5F show that donor cone photoreceptors mature more rapidly in the recipient subretinal space. FIG. 5A shows UMAP embedding the maturation trajectories in pseudotime of cone photoreceptors in transplanted (n=3) and cultured retinal organoids (age-matched, n=2), compared to normal in vivo human cone development (aged from embryonic week nine to adulthood). Cells are colored by cell type (top UMAP) and pseudotime (bottom UMAP). FIG. 5B shows the ridge plot showing that the transcriptional maturation of transplanted cone photoreceptors resemble adult human cone photoreceptors, whereas cultured cone photoreceptors resemble embryonic human cone photoreceptors (aged 9-18 embryonic weeks). FIG. 5C shows ScRNA-seq violin plots showing the upregulation of OPN1LW, OPN1MW and OPN1SW in transplanted retinal organoids compared to cultured retinal organoids. FIG. 5D shows IHC staining and quantification showing significantly more L/Mopsin⁺ and S-opsin⁺ cone photoreceptors in transplanted than cultured retinal organoids (n=16-20 sections from four individual samples per group). FIG. 5E shows IHC images showing representative L/Mopsin⁺ or S-opsin⁺ cone photoreceptors with (OS⁺, yellow arrow heads) or without (OS⁻) outer segments. Histological quantification of the fraction of L/M-opsin⁺ or S-opsin⁺ cells with inner/outer segment formation (Segment⁺) was significantly greater in transplanted than cultured retinal organoids (n=16-20 sections from four individual samples/group). FIG. 5F show single-cell patch-clamp recording of a transplanted human cone showed large-capacitance current.

FIGS. 6A-6E show that donor rod photoreceptors mature more rapidly in the recipient subretinal space. FIG. 6A shows UMAP embedding the maturation trajectories of rod photoreceptors in transplanted (n=3) and age-matched cultured retinal organoids (n=2), compared to in vivo human rod development (aged from embryonic week nine to adult). Cells were colored by cell type (top UMAP) and pseudotime (bottom UMAP). FIG. 6B shows the ridgeline plot indicating that the transcriptional maturation of transplanted rod photoreceptors resemble adult human rod photoreceptors, whereas cultured rod photoreceptors transcriptionally resemble embryonic human rod photoreceptors (aged 9-27 embryonic weeks). FIG. 6C show ScRNA-seq analysis showing upregulation of RHO gene expressions in transplanted retinal organoids compared to cultured retinal organoids. FIG. 6D show IHC staining and quantification showing greater proportion of Rho⁺ photoreceptors in transplanted than cultured Crx:tdTomato⁺ photoreceptors (n=20 sections from four individual samples/group. FIG. 6E shows IHC staining showing representative Rho⁺ rod photoreceptors with (OS⁺, yellow arrow heads) or without (OS⁻) outer segments. Histological quantification showed a greater fraction of rods with inner/outer segment formation (Segment⁺) in transplanted than cultured retinal organoids (n=20 sections from four individual samples/group).

FIGS. 7A-7C show breeding and phenotyping of the recipient Rd1/NS mice. FIG. 7A illustrates the schematic showing the recipient Rd1/NS mice that were generated by crossbreeding the Rd1 (Pde6brd1) and NOD/SCID (NOD.CB17-Prkdcscid/J) mice. FIG. 7B shows IHC staining showing the comparable phenotype of retinal degeneration in age-matched Rd1/NS and Rd1 mice: the outer nuclear layer was lost in both and there was no expression of L/M-opsin, S-opsin, and Rhodopsin (Rho). C57.BL/6J mice served as wild-type controls. FIG. 7C shows flow cytometry analysis showing the deficiency of CD3⁺ T cells and CD45R⁺ B cells in Rd1/NS mice, corresponding to the NOD/SCID genotype. C57.BL/6J mice served as wild-type controls.

FIG. 8 shows RNAscope staining of positive and negative control probes. Cryosections of non-transplanted Rd1/NS mice and cultured retinal organoids were stained with 3-plex positive and negative control probes in combination with TSA-Cy3 or TSA-Cy5 fluorophores. The positive staining showed the expression of positive control genes PPIB (Cy3) and POLR2A (Cy5)

FIGS. 9A-9D show quality control of the scRNA-Seq data. FIGS. 9A and 9B show the number of genes and unique molecular identifiers per cell. Each bar is a cell and is colored by the sample library and ordered along the x-axis in descending order. FIG. 9C shows UMAP plot showing cells colored by sample library. FIG. 9D shows UMAP plot showing 10 (0-9) transcriptionally distinct cell clusters.

FIGS. 10A-10C show UMAPs of migration and proliferation cell clusters. FIG. 10A shows the UMAP distinguishing the cell clusters of cultured (grey) and transplanted retinal organoids (purple). FIG. 10B shows the UMAP colored cell clusters gaining transcriptomic characteristics of both migration and proliferation. FIG. 10C shows the UMAPs displayed the expression of marker genes in different cell subpopulations of transplanted and cultured retinal organoids.

FIGS. 11A-11B show individual cell clusters of pseudotime analysis and marker gene expression of cone and rod photoreceptors in cultured and transplanted retinal organoids. FIG. 11A shows the UMAP identifying individual cell clusters of human retinae (aged from embryonic week 9 to adult, cone developmental dataset: n=7,654 cells; rod developmental dataset: n=25,186 cells), cultured retinal organoids (cone: n=1,639 cells, rod: n=1,469 cells), and transplanted retinal organoids (cone: n=210 cells, rod: n=504 cells). FIG. 11B shows the heatmap demonstrating the upregulation of marker genes specific for cone and rod photoreceptors in transplanted retinal organoids (including three independent replicates “Transplanted-1, Transplanted-2, Transplanted-3”), compared to cultured retinal organoids (including two independent replicates “Cultured-1, Cultured-2”).

FIGS. 12A-12B show identification and quantitation of pre-synaptic markers in cultured and transplanted retinal organoids. FIG. 12A shows the heatmaps showing some of the synaptic genes were upregulated in the transplanted retinal organoids (including cones and rods) compared to the cultured retinal organoids. FIG. 12B shows IHC staining and quantification demonstrating that CtBP2⁺ synaptic ribbons in photoreceptors (CRX:tdTomato⁺) were significantly more in transplanted than cultured retinal organoids. IHC staining of SCGN (green) was performed to indicate the recipient bipolar layer. The anti-human nuclear antibody (HNA, blue) was adopted to label human cells. Abbreviation: INL: inner nuclear layer.

FIGS. 13A-13B show single cell RNA sequencing of prioritized CD markers. FIGS. 13A and 13B show CD302 highly expressed in astrocytes, and brain and spinal cord like cells (BSLCs). CD9 is highly expressed in non-neuronal cells and BSLCs. CD99 is highly expressed in all non-neuronal cells and BSLCs. CD24 is highly expressed in amacrine cells, photoreceptor precursor cells, mature bipolar cells, horizontal cells, and retinal ganglion cells. The data are in support of a strategy wherein enrichment based on negative selection for CD24, CD302, CD9, CD99 singly or in combination will result in an enriched population of photoreceptor cells that are therapeutically competent for cellular components transfer into acceptor cells of the recipient retina.

### DETAILED DESCRIPTION

The present disclosure provides methods for generating and sorting retinal cells for use in cellular component transfer therapy, sorted populations of retinal cells generated by such methods, and compositions comprising sorted populations of retinal cells. The present disclosure also provides uses of the sorted populations of retinal cells and compositions comprising thereof for preventing and/or treating inherited or acquired retinal degenerative diseases.

Non-limiting embodiments of the presently disclosed subject matter are described by the present specification and Examples. For purposes of clarity of disclosure and not by way of limitation, the detailed description is divided into the following subsections:
1. Definitions;
2. Methods of Generating Retinal Cells;
3. Retinal Cell Populations & Retinal Cell Compositions; and
4. Methods of Treating Inherited Retinal Degenerative Diseases.

### 1. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the compositions and methods of the present disclosure and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type, such as a population of photoreceptor cells, or a population of undifferentiated stem cells. Alternatively, the population may comprise more than one cell type, for example a mixed cell population. In certain embodiments, the cells in the population of cells are entirely dissociated from each other, e.g., the population of cells is a suspension of individual cells. In certain embodiments, the population of cells comprises undissociated clusters of cells. For example, but not by way of limitation, such populations of cells can comprise up to about 1%, up to about 2%, up to about 3%, up to about 4%, up to about 5%, up to about 6%, up to about 7%, up to about 8%, up to about 9%, or up to about 10% of the cells in the population present as undissociated clusters comprising up to about 10 cells. In certain embodiments, such populations of cells can comprise up to about 1%, up to about 2%, up to about 3%, up to about 4%, up to about 5%, up to about 6%, up to about 7%, up to about 8%, up to about 9%, or up to about 10% of cells in the population present as undissociated clusters comprising up to about 25 cells.

As used herein, the term “stem cell” refers to a cell with the ability to divide for indefinite periods in culture and to give rise to specialized cells.

As used herein, the term “embryonic stem cell” and “ESC” refer to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human embryo. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “totipotent” refers to an ability to give rise to all the cell types of the body plus all of the cell types that make up the extraembryonic tissues such as the placenta.

As used herein, the term “multipotent” refers to an ability to develop into more than one cell type of the body.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell formed by the introduction of certain embryonic genes (such as but not limited to OCT4, SOX2, and KLF4 transgenes) (see, for example, Takahashi and Yamanaka Cell 126, 663-676 (2006), herein incorporated by reference) into a somatic cell.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self-renewal (in the laboratory) and differentiation.

As used herein, the term “proliferation” refers to an increase in cell number.

As used herein, the term “undifferentiated” refers to a cell that has not yet developed into a specialized cell type.

As used herein, the term “differentiation” refers to a process whereby an unspecialized embryonic cell acquires the features of a specialized cell such as a retinal, heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as a retinal cell. In references to a stem cell, “directed differentiation” refers to the use of small molecules, growth factor proteins, and other growth conditions to promote the transition of a stem cell from the pluripotent state into a more mature or specialized cell fate.

As used herein, the term “inducing differentiation” in reference to a cell refers to changing the default cell type (gene expression profile and/or phenotype) to a non-default cell type (gene expression profile and/or phenotype). Thus, “inducing differentiation in a stem cell” refers to inducing the stem cell (e.g., human stem cell) to divide into progeny cells with characteristics that are different from the stem cell, such as in gene expression profile (e.g., change in gene expression as determined by genetic analysis such as a microarray) and/or phenotype (e.g., change in the number or presence of a protein marker, e.g., a cell surface marker, of rod or cone photoreceptor cells, such as CRX, RCVRN, CNGA3, CNGB3, ARR3, THRB, OPN1S2, OPN1MW, NRL, NR2E3, PDE6B, CNGA1, and RHO).

As used herein, the term “sorting” refers to positive sorting, i.e., where the presence of a particular feature results in inclusion of a cell in a sorted population, as well as negative sorting, i.e., where the presence of a particular feature results in the exclusion of the cell from the sorted population. Exemplary features associated with positive sorting as disclosed herein include, but are not limited to, cellular markers of photoreceptor cells, e.g., CD73. Exemplary features associated with negative sorting as disclosed herein include, but are not limited to, cellular markers of non-photoreceptor cells, e.g., CD24, CD302, CD9, and CD99. Exemplary features associated with negative sorting as disclosed herein can also include, but are not limited to, cellular markers of astrocytes, cellular markers of brain and spinal cord-like (BSL) cells, as well as both markers for astrocytes and markers for BSL cells. Sorted populations can include positively sorted cells, negatively sorted cells, or combinations of positively and negatively sorted cells.

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, a spinner flask, and the like, and contains nutrients to nourish and support the cells. Culture medium may also include growth factors added to produce desired changes in the cells.

As used herein, the term “contacting” a cell or cells with a compound (e.g., at least one inhibitor, activator, and/or inducer) refers to providing the compound in a location that permits the cell or cells access to the compound. The contacting may be accomplished using any suitable method. For example, contacting can be accomplished by adding the compound, in concentrated form, to a cell or population of cells, for example in the context of a cell culture, to achieve the desired concentration. Contacting may also be accomplished by including the compound as a component of a formulated culture medium.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, retina formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) an ultimate parent cell in a cell line, tissue (such as a dissociated embryo, or fluids using any manipulation, such as, without limitation, single cell isolation, culture in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease, a treatment can prevent deterioration due to a disease in an affected or diagnosed subject or a subject suspected of having the disease, but also a treatment may prevent the onset of the disease or a symptom of the disease in a subject at risk for the disease or suspected of having the disease.

### 2. Methods of Generating and Sorting Retinal Cells

### 2.1. Three-Dimensional Cell Culture of Retinal Cells

The present disclosure provides for in vitro methods for inducing differentiation of stem cells (e.g., human stem cells) and subsequently sorting the resulting differentiated cells. For example, the presently disclosed subject matter provides in vitro methods for inducing differentiation of stem cells to produce retinal cells, e.g., rod and/or cone photoreceptor cells, and subsequently sorting said retinal cells. In certain embodiments, the stem cells are pluripotent stem cells. In certain embodiments, the pluripotent stem cells are selected from embryonic stem cells (ESCs), induced pluripotent stem cells (iPSCs), and combinations thereof. In certain embodiments, the stem cells are multipotent stem cells. Non-limiting examples of stem cells that can be used with the presently disclosed methods include human, nonhuman primate or rodent nonembryonic stem cells, embryonic stem cells, induced nonembryonic pluripotent cells and engineered pluripotent cells. In certain embodiments, the stem cells are human stem cells. Non-limiting examples of human stem cells include human pluripotent stem cell (hPSC) (including, but not limited to human embryonic stem cells (hESC) and human induced pluripotent stem cells (hiPSC)), human parthenogenetic stem cells, primordial germ cell-like pluripotent stem cells, epiblast stem cells, F-class pluripotent stem cells, somatic stem cells, cancer stem cells, or any other cell capable of lineage specific differentiation. In certain embodiments, the stem cell is an embryonic stem cell (ESC). In certain embodiments, the stem cell is a human embryonic stem cell (hESC). In certain embodiments, the stem cell is an induced pluripotent stem cell (iPSC). In certain embodiments, the stem cell is a human induced pluripotent stem cell (hiPSC).

In certain embodiments, the in vitro methods for inducing differentiation of stem cells to produce retinal cells of the present disclosure comprise the use of factors that promote rod and cone photoreceptor fate specification and survival. In certain embodiments, the in vitro methods for inducing differentiation of stem cells to produce retinal cells of the present disclosure comprise the use of factors that that suppress fate specification and survival of retinal interneurons, e.g., bipolar cells and retinal ganglion cells. In certain embodiments, the in vitro methods for inducing differentiation of stem cells to produce retinal cells of the present disclosure comprise the use of factors that that suppress fate specification and survival of retinal glia, e.g., Muller glia. In certain embodiments, the in vitro methods for inducing differentiation of stem cells to produce retinal cells of the present disclosure comprise the use of factors that that: (a) promote rod and cone photoreceptor fate specification and survival; suppress fate specification and survival of retinal interneurons, e.g., bipolar cells and retinal ganglion cells; and/or (c) suppress fate specification and survival of retinal glia, e.g., Muller glia.

In certain embodiments, the present disclosure is directed to the generation of three-dimensional retinal organoids, e.g., three dimensional human retinal organoids. For example, but not by way of limitation, the strategies for generating three-dimensional human retinal organoids can be employed as described in Eldred et al., Science, 362:6411 (2018); Zhong et al., Nat Commun., 5:4047 (2014); Reichman et al., Stem Cells, 35:1176-88 (2017); Wahlin et al., Sci Rep., 7:766 (2017); Hallam et al., Stem Cells, 36:1535-51 (2018); Kaya et al., Mol. Vis., 25:663-678 (2019); or Regent et al., Mol Vis., 26:97-105 (2020), each of which is incorporated herein by reference in its entirety. In certain embodiments, human retinal organoids are differentiated to achieve specific ratios of cone subtypes (red/Long, green/Medium, and blue/Short). For example, but not by way of limitation, culturing the organoid in the presence of low retinoic acid (RA), e.g., less than about 1 μM RA, leads to organoids having high red cones. In certain exemplary embodiments, culturing the organoid in high RA, e.g., greater than about 1 μM to about 20 μM RA, (or Knockout of CYP26a1) leads to organoids with high blue and green cones. In certain exemplary embodiments, culturing the organoid in RA through day 80 leads to a peripheral mix of red, green, and blue cones. In certain exemplary embodiments, culturing the organoid in high thyroid hormone (T3), e.g., greater than about 1 nM to about 1 μM T3, with high RA e.g., greater than about 1 μM to about 20 μM RA, leads to organoids with high green cones. In certain exemplary embodiments, culturing the organoid in high T3, e.g., greater than about 1 nM to about 1 μM T3, with low RA, e.g., less than about 1 μM RA, leads to organoids with high red cones. In certain exemplary embodiments, knock out of thyroid hormone receptor in the organoid leads to high blue cones.

In certain embodiments, the differentiation of stem cells to retinal organoids includes in vitro differentiation of stem cells to cells expressing at least one retinal organoid marker. In certain embodiments, the differentiation of stem cells to retinal organoids includes in vitro differentiation of stem cells to cells exhibiting at least one morphological characteristic associated with retinal organoid differentiation. In certain embodiments, the differentiation of stem cells to retinal organoids includes in vitro differentiation of stem cells to cells expressing at least one retinal organoid marker and exhibiting at least one morphological characteristic associated with retinal organoid differentiation. Non-limiting examples of retinal organoid markers include Nrl, Rho, Arr3, and combinations thereof. Non-limiting examples of retinal organoid morphological characteristics include: (a) the development of a multilayered retinal organoid anatomy comprising, e.g., a photoreceptor outer nuclear layer and nascent outer segments; and (b) retinal pigment epithelium (RPE) pigmentation development.

In certain embodiments, the stem cells are allowed to differentiate to attain a target differentiation stage of the cells of the retinal organoid of at least about 45 days to about 300 days. In certain embodiments, the stem cells are allowed to differentiate to attain a target differentiation stage of the cells of the retinal organoid of at least about 50 days to about 300 days. In certain embodiments, the stem cells are allowed to differentiate to attain a target differentiation stage of the cells of the retinal organoid of at least about 55 days to about 300 days. In certain embodiments, the stem cells are allowed to differentiate to attain a target differentiation stage of the cells of the retinal organoid of at least about 60 days to about 300 days. In certain embodiments, the stem cells are allowed to differentiate to attain a target differentiation stage of the cells of the retinal organoid of at least about 70 days to about 300 days. In certain embodiments, the stem cells are allowed to differentiate to attain a target differentiation stage of the cells of the retinal organoid of at least about 75 days to about 300 days. In certain embodiments, the stem cells are allowed to differentiate to attain a target differentiation stage of the cells of the retinal organoid of at least about 80 days to 300 days. In certain embodiments, the stem cells are allowed to differentiate to attain a target differentiation stage of the cells of the retinal organoid of at least about 85 days to about 300 days. In certain embodiment, the stem cells are allowed to differentiate to attain a target differentiation stage of the cells of the retinal organoid of at least about 90 days, at least about 91 days, at least about 93 days, at least about 94 days, at least about 95 days, at least about 96 days, at least about 97 days, at least about 98 days, at least about 99 days, at least about 100 days, at least about 101 days, at least about 102 days, at least about 103 days, at least about 104 days, at least about 105 days, at least about 106 days, at least about 107 days, at least about 108 days, at least about 109 days, at least about 110 days, at least about 111 days, at least about 112 days, at least about 113 days, at least about 114 days, at least about 115 days, at least about 116 days, at least about 117 days, at least about 118 days, at least about 119 days, at least about 120 days, at least about 121 days, at least about 122 days, at least about 123 days, at least about 124 days, at least about 125 days, at least about 126 days, at least about 128 days, at least about 129 days, at least about 130 days, at least about 131 days, at least about 132 days, at least about 133 days, at least about 134 days, at least about 135 days, at least about 136 days, at least about 137 days, at least about 138 days, at least about 139 days, or at least about 140 days. The duration of differentiation can be noted as “DD”, e.g., allowed cells to differentiate to attain a target differentiation stage of the cells of the retinal organoid of at least about 50 days (“DD50”) to about 300 days (“DD300”).

### 2.2. Dissociation of Retinal Organoids

In certain embodiments, the present disclosure is directed to the generation of populations of retinal cells via the dissociation of the above-described retinal organoids. In certain embodiments, such dissociation involves the disruption of the laminar organization of cells in the organoid. In certain embodiments, such retinal organoids are dissociated by the addition of specific enzymes and/or additives that ensure that the cells remain in dissociated cell suspension rather than as aggregates. For example, but not by way of limitation, enzymes useful in connection with the dissociation of retinal organoids include papain and trypsin. Compositions useful in ensuring that the cells remain in a dissociated cell suspension include compositions comprising DNAse. Compositions useful to enhance the survival of the cells in a dissociated cell suspension include compositions comprising a B-27 cell culture supplement (Thermo Fisher Scientific) or an N-2 cell culture supplement (Thermo Fisher Scientific).

In certain embodiments, the populations of retinal cells resulting from dissociation of the retinal organoids of the present disclosure will contain at least 70% single cells, relative to the total number of cells (including doublet cells, triplet cells, and larger order undissociated clusters of cells). In certain embodiments, the cell populations of the present disclosure will contain between 70%-80% single cells, relative to the total number of cells (including doublet cells, triplet cells, and larger order undissociated clusters of cells). In certain embodiments, the cell populations of the present disclosure will contain between 70%-85% single cells, relative to the total number of cells (including doublet cells, triplet cells, and larger order undissociated clusters of cells). In certain embodiments, the cell populations of the present disclosure will contain between 70%-90% single cells, relative to the total number of cells (including doublet cells, triplet cells, and larger order undissociated clusters of cells). In certain embodiments, the cell populations of the present disclosure will contain between 70%-95% single cells, relative to the total number of cells (including doublet cells, triplet cells, and larger order undissociated clusters of cells). In certain embodiments, the cell populations of the present disclosure will contain between 70%-100% single cells, relative to the total number of cells (including doublet cells, triplet cells, and larger order undissociated clusters of cells).

In certain embodiments, the retinal cell populations resulting from dissociation of the retinal organoids of the present disclosure will contain at least 80% single cells, relative to the total number of cells (including doublet cells, triplet cells, and larger order undissociated clusters of cells). In certain embodiments, the cell populations of the present disclosure will contain between 80%-85% single cells, relative to the total number of cells (including doublet cells, triplet cells, and larger order undissociated clusters of cells). In certain embodiments, the cell populations of the present disclosure will contain between 80%-90% single cells, relative to the total number of cells (including doublet cells, triplet cells, and larger order undissociated clusters of cells). In certain embodiments, the cell populations of the present disclosure will contain between 80%-95% single cells, relative to the total number of cells (including doublet cells, triplet cells, and larger order undissociated clusters of cells). In certain embodiments, the cell populations of the present disclosure will contain between 80%-100% single cells, relative to the total number of cells (including doublet cells, triplet cells, and larger order undissociated clusters of cells).

In certain embodiments, the retinal cell populations resulting from dissociation of the retinal organoids of the present disclosure will contain at least 85% single cells, relative to the total number of cells (including doublet cells, triplet cells, and larger order undissociated clusters of cells). In certain embodiments, the cell populations of the present disclosure will contain between 85%-90% single cells, relative to the total number of cells (including doublet cells, triplet cells, and larger order undissociated clusters of cells). In certain embodiments, the cell populations of the present disclosure will contain between 85%-95% single cells, relative to the total number of cells (including doublet cells, triplet cells, and larger order undissociated clusters of cells). In certain embodiments, the cell populations of the present disclosure will contain between 85%-100% single cells, relative to the total number of cells (including doublet cells, triplet cells, and larger order undissociated clusters of cells).

In certain embodiments, the retinal cell populations resulting from dissociation of the retinal organoids of the present disclosure will contain at least 90% single cells, relative to the total number of cells (including doublet cells, triplet cells, and larger order undissociated clusters of cells). In certain embodiments, the cell populations of the present disclosure will contain between 90%-95% single cells, relative to the total number of cells (including doublet cells, triplet cells, and larger order undissociated clusters of cells). In certain embodiments, the cell populations of the present disclosure will contain between 90%-100% single cells, relative to the total number of cells (including doublet cells, triplet cells, and larger order undissociated clusters of cells).

In certain embodiments, the retinal cell populations resulting from dissociation of the retinal organoids of the present disclosure will contain at least 95% single cells, relative to the total number of cells (including doublet cells, triplet cells, and larger order undissociated clusters of cells). In certain embodiments, the cell populations of the present disclosure will contain between 95%-100% single cells, relative to the total number of cells (including doublet cells, triplet cells, and larger order undissociated clusters of cells).

### 3. Retinal Cell Populations & Retinal Cell Compositions

In certain embodiments, the present disclosure is directed to the generation and sorting of retinal cell populations. In certain embodiments, the retinal cell populations are sorted, e.g., via fluorescence-activated cell sorting, to selectively enrich for and/or negatively select for specific cell types.

In certain embodiments, the positively sorted cells of the sorted retinal cell populations of the present disclosure express a marker of photoreceptor cells, e.g., CD73. In certain embodiments, the negatively sorted cells excluded from the sorted retinal cell populations of the present disclosure express a marker of non-photoreceptor cells, e.g., CD24, CD302, CD9, and CD99.

In certain embodiments, the negatively sorted cells excluded from the sorted retinal cell populations of the present disclosure express a marker that distinguishes non-photoreceptor cells from photoreceptor cells. For example, but not by way of limitation, such negative sorting can be performed on the basis of the expression of one or more cell surface marker. In certain embodiments, such cell surface marker(s) can be selected from CD markers and surface receptors. In certain embodiments, such marker(s) will exhibit >75% expression in non-photoreceptor cells and <25% expression in photoreceptor cells. For example, but not by way of limitation, such negative sorting can be performed on the basis of the expression of one or more of the following: ITM2B; CD63; ENO1; CALR; CANX; CLU; SLC3A2; BSG; GPM6B; ITGB1; PTTG1IP; TIMP1; PMEPA1; SSR2; DKK3; LRP1; ATRAID; HLA-A; HLA-C; EMP3; TMED9; GOLIM4; LTBP3; GALNT1; CD151; PLD3; CALU; LSAMP; CD59; SLC2A1; LAMP2; HLA-B; COL11A1; DPP7; DCBLD2; CD164; SLC1A3; F3; CTSD; FLNA; SLC39A10; FN1; TMEM106C; TMEM179B; ATP1B3; HLA-E; TMEM132A; FLT1; FGFR1; CAPNS1; FAT1; ANGPTL1; LRP10; CRELD2; SPPL2A; TSPAN4; PRSS35; ECE1; SYPL1; SORCS2; COL2A1; DNER; COL6A1; CD44; GPC1; PCDH9; CRIM1; CHL1; TTYH3; IKBIP; NECTIN2; FBLN2; CCDC80; DAG1; PBXIP1; PRSS23; ACAA1; NTRK2; FSTL1; BCHE; TNFRSF1A; LGALS3BP; ITGB8; CP; ADGRG1; VCAN; OLFM1 NRP1; SCARB1; TSPAN6; PCDH17; PLTP; NECTIN3; PTPRD; CADM4; UNC5B; CSPG5; AXL; PLXNB2; PLPP3; NOTCH2; SLITRK2; AEBP1; ANGPTL4; COLEC12; VAMP5; NLGN4X; FGFRL1; EFNB2; COL5A1; LAMB2; LAMC1; IGFBP3; FNDC5; FCGRT; ADRA2C; SERPING1; EPHB2; CDH11; COL1A2; CNTFR; AGRN; ROBO1; LOX; MRC2; COL6A2; SLC6A11; DSC2; IGSF8; EMP1; ABI3BP; TTYH2; NOTCH1; ANO6; A2M; SORCS1; EFNA1; PTPRG; TF; EMP2; CEMIP2; SERPINE2; CDON; EGFR; PCDH7; MAN2A1; IL1R1; COL1A1; SEMA5A; ANTXR1; S1PR3; ITPRIP; MXRA8; PRELP; AQP1; CSF1; BCAN; ADGRA2; CA12; FAT3; HEPACAM; FGFR3; TRIL; HSD17B2; and HP. In certain embodiments, the specific subset of non-photoreceptor cell markers used in negatively sorting non-photoreceptor cells from photoreceptor cells is one or more of the following markers: DKK3; LRP1; CLU; PMEPA1; ITGB1; and PTTG1IP.

In certain embodiments, the negatively sorted cells excluded from the sorted retinal cell populations of the present disclosure express a marker that distinguishes astrocytes from photoreceptor cells. For example, but not by way of limitation, such negative sorting can be performed on the basis of the expression of one or more cell surface marker. In certain embodiments, such cell surface marker(s) can be selected from CD markers and surface receptors. In certain embodiments, such marker(s) will exhibit >75% expression in astrocytes and <25% expression in photoreceptor cells. For example, but not by way of limitation, such negative sorting can be performed on the basis of the expression of one or more of the following: ITM2B; CD63; ENO1; CALR; CANX; CLU; SLC3A2; BSG; GPM6B; ITGB1; PTTG1IP; TIMP1; PMEPA1; SSR2; DKK3; LRP1; ATRAID; HLA-A; HLA-C; EMP3; TMED9; GOLIM4; LTBP3; GALNT1; CD151; PLD3; CALU; LSAMP; CD59; SLC2A1; LAMP2; HLA-B; COL11A1; DPP7; DCBLD2; CD164; SLC1A3; F3; CTSD; FLNA; SLC39A10; FN1; TMEM106C; TMEM179B; ATP1B3; HLA-E; TMEM132A; FLT1; FGFR1; CAPNS1; FAT1; ANGPTL1; LRP10; CRELD2; SPPL2A; TSPAN4; PRSS35; ECE1; SYPL1; SORCS2; COL2A1; DNER; COL6A1; CD44; GPC1; PCDH9; CRIM1; CHL1; TTYH3; IKBIP; NECTIN2; FBLN2; CCDC80; DAG1; PBXIP1; PRSS23; ACAA1; NTRK2; FSTL1; BCHE; TNFRSF1A; LGALS3BP; ITGB8; CP; ADGRG1; VCAN; OLFM1; NRP1; SCARB1; TSPAN6; PCDH17; PLTP; NECTIN3; PTPRD; CADM4; UNC5B; CSPG5; AXL; PLXNB2; PLPP3; NOTCH2; SLITRK2; AEBP1; ANGPTL4; COLEC12; VAMP5; NLGN4X; FGFRL1; EFNB2; COL5A1; LAMB2; LAMC1; IGFBP3; FNDC5; FCGRT; ADRA2C; SERPING1; EPHB2; CDH11; COL1A2; CNTFR; AGRN; ROBO1; LOX; MRC2; COL6A2; SLC6A11; DSC2; IGSF8; EMP1; ABI3BP; TTYH2; NOTCH1; ANO6; A2M; SORCS1; EFNA1; PTPRG; TF; EMP2; CEMIP2; SERPINE2; CDON; EGFR; PCDH7; MAN2A1; IL1R1; COL1A1; SEMA5A; ANTXR1; SIPR3; ITPRIP; MXRA8; PRELP; AQP1; CSF1; BCAN; ADGRA2; CA12; FAT3; HEPACAM; FGFR3; TRIL; HSD17B2; and HP. In certain embodiments, the specific subset of astrocyte markers used in negatively sorting astrocytes from photoreceptor cells is one or more of the following markers: ADGRL4; SERPINE2; BCHE; ABI3BP; NRP1; FSTL1; FAT1; NTRK2; FBLN2; PRSS35; SLC1A3; FCGRT; LAMC1; TF; SORCS2; DKK3; LRP1; PTPRD; ANGPTL1; LTBP3; CLU; CNTNAP2; CD151; PCDH9; CRIM1; CSPG5; and PMEPA1.

In certain embodiments, the negatively sorted cells excluded from the sorted retinal cell populations of the present disclosure express a marker that distinguishes “brain and spinal cord-like” (BSL) cells, i.e., those BSL cells as described in Example 1, from photoreceptor cells. For example, but not by way of limitation, such negative sorting can be performed on the basis of the expression of one or more cell surface marker. In certain embodiments, such cell surface marker(s) can be selected from CD markers and surface receptors. In certain embodiments, such marker(s) will exhibit >75% expression in BSL cells and <25% expression in photoreceptor cells. For example, but not by way of limitation, such negative sorting can be performed on the basis of the expression of one or more of the following: CLU; ITM2B; PTPRZ1; GPM6B; ATP1B2; CD63; BCAN; SLC1A3; SERPINE2; LRP1; PTPRA; ADGRG1; ENO1; CANX; SLC3A2; DNER; PTTG1IP; CALR; PCDH9; CCDC80; LSAMP; HEPACAM; F3; PLPP3; APLP2; FBLN2; TIMP1; SLC6A11; CSPG5; JAM2; FGFR3; DKK3; GOLIM4; NCAM1; CHL1; NRCAM; HLA-A; TMEM132A; PMEPA1; ITGAV; SSR2; ACAA1; BCHE; CD59; FAT3; PCDH17; ST3GAL5; PBXIP1; LAMP1; ITGB1; HP; ITGB8; SGCB; LAMP2; CLDND1; TMEM106B; PTCH1; PLTP; RNF13; HLA-C; PTPRD; TMEM30A; TRIL; RAB5C; TTYH3; DAG1; CADM4; UBA1; SLC6A9; LRRC8A; ATP1B3; SPPL2A; NTRK2; RNF130; LIFR; EMP3; PCDH7; NTRK3; COL6A1; IL17D; LRP10; ADAM19; SGCE; FAT1; SLC44A1; LTBP3; SLC39A10; ABCA1; SYPL1; SLITRK2; GNPTG; CD302; MRC2; LRP4; CALU; CD151; SORL1; TSPAN6; LRRN1; TENM2; CAPNS1; NLGN1; SLC15A2; NLGN4X; EGFR; ADORA1; SLC9A7; SIRPA; EFCAB14; ANGPTL1; FGFR1; VAMP5; CLDN12; LAMB2; GPR155; FGFR2; SLC44A2; LRP1B; PTGFRN; FNDC5; NOTCH1; DPY19L4; S1PR1; CD44; TNFRSF1A; FAM234A; CDH4; HLA-E; COL11A1; NCAM2; AQP1; CPQ; EMP1; FCGRT; GPC5; ROBO1; VCAN; LGALS3BP; LDLR; LRRC4B; NOTCH2; ALCAM; RYR3; SLC9A9; TMEM94; VCAM1; IGSF1; PCDHA10; CDH10; CACHD1; P2RX7; AEBP1; PLXNB1; AXL; ALPL; ST3GAL4; SERPINI1; ITGB5; CD58; FGFRL1; PLPP1; TTYH2; IL17RB; FAM171A1; IL17RD; ANO6; ADAM22; PTPRG; ANTXR1; ZDHHC23; AGRN; COL14A1; POSTN; CNTFR; SEMA5A; FLNA; EMP2; TFPI; ITGA7; MXRA8; TENM4; FSTL1; CD82; NRP2; GPC4; ARSF; LAMC1; KIT; SEMA4A; LTBP1; and CSF1. In certain embodiments, the specific subset of BSL cell markers used in negatively sorting BSL cells from photoreceptor cells is one or more of the following markers: HEPACAM; FGFR3; SERPINE2; BCAN; CCDC80; PLPP3; CHL1; ADGRG1; SLC6A11; LSAMP; FBLN2; F3; SLC1A3; DKK3; LRP1; DNER; CLU; PCDH9; and CSPG5.

In certain embodiments, the negatively sorted cells excluded from the sorted retinal cell populations of the present disclosure express a marker that distinguishes astrocytes from photoreceptor cells and the cells are further negatively sorted (either before, after, or concurrently with the negative astrocyte sorting) based on expression of a marker that distinguishes BLS cells from photoreceptor cells. For example, but not by way of limitation, such negative sorting can be performed on the basis of the expression of one or more cell surface marker. In certain embodiments, such cell surface marker(s) can be selected from CD markers and surface receptors. In certain embodiments, such marker(s) will exhibit >75% expression in astrocytes and <25% expression in photoreceptor cells for the marker(s) employed for negative astrocyte sorting and such marker(s) will exhibit >75% expression in BSL cells and <25% expression in photoreceptor cells for the marker(s) employed for negative BSL sorting. For example, but not by way of limitation, such negative sorting can be performed on the basis of the expression of one or more of the following astrocyte markers: ITM2B; CD63; ENO1; CALR; CANX; CLU; SLC3A2; BSG; GPM6B; ITGB1; PTTG1IP; TIMP1; PMEPA1; SSR2; DKK3; LRP1; ATRAID; HLA-A; HLA-C; EMP3; TMED9; GOLIM4; LTBP3; GALNT1; CD151; PLD3; CALU; LSAMP; CD59; SLC2A1; LAMP2; HLA-B; COL11A1; DPP7; DCBLD2; CD164; SLC1A3; F3; CTSD; FLNA; SLC39A10; FN1; TMEM106C; TMEM179B; ATP1B3; HLA-E; TMEM132A; FLT1; FGFR1; CAPNS1; FAT1; ANGPTL1; LRP10; CRELD2; SPPL2A; TSPAN4; PRSS35; ECE1; SYPL1; SORCS2; COL2A1; DNER; COL6A1; CD44; GPC1; PCDH9; CRIM1; CHL1; TTYH3; IKBIP; NECTIN2; FBLN2; CCDC80; DAG1; PBXIP1; PRSS23; ACAA1; NTRK2; FSTL1; BCHE; TNFRSF1A; LGALS3BP; ITGB8; CP; ADGRG1; VCAN; OLFM1; NRP1; SCARB1; TSPAN6; PCDH17; PLTP; NECTIN3; PTPRD; CADM4; UNC5B; CSPG5; AXL; PLXNB2; PLPP3; NOTCH2; SLITRK2; AEBP1; ANGPTL4; COLEC12; VAMP5; NLGN4X; FGFRL1; EFNB2; COL5A1; LAMB2; LAMC1; IGFBP3; FNDC5; FCGRT; ADRA2C; SERPING1; EPHB2; CDH11; COL1A2; CNTFR; AGRN; ROBO1; LOX; MRC2; COL6A2; SLC6A11; DSC2; IGSF8; EMP1; ABI3BP; TTYH2; NOTCH1; ANO6; A2M; SORCS1; EFNA1; PTPRG; TF; EMP2; CEMIP2; SERPINE2; CDON; EGFR; PCDH7; MAN2A1; IL1R1; COL1A1; SEMA5A; ANTXR1; S1PR3; ITPRIP; MXRA8; PRELP; AQP1; CSF1; BCAN; ADGRA2; CA12; FAT3; HEPACAM; FGFR3; TRIL; HSD17B2; and HP; and on the basis of the expression of one or more of the following BSL cell markers: CLU; ITM2B; PTPRZ1; GPM6B; ATP1B2; CD63; BCAN; SLC1A3; SERPINE2; LRP1; PTPRA; ADGRG1; ENO1; CANX; SLC3A2; DNER; PTTG1IP; CALR; PCDH9; CCDC80; LSAMP; HEPACAM; F3; PLPP3; APLP2; FBLN2; TIMP1; SLC6A11; CSPG5; JAM2; FGFR3; DKK3; GOLIM4; NCAM1; CHL1; NRCAM; HLA-A; TMEM132A; PMEPA1; ITGAV; SSR2; ACAA1; BCHE; CD59; FAT3; PCDH17; ST3GAL5; PBXIP1; LAMP1; ITGB1; HP; ITGB8; SGCB; LAMP2; CLDND1; TMEM106B; PTCH1; PLTP; RNF13; HLA-C; PTPRD; TMEM30A; TRIL; RAB5C; TTYH3; DAG1; CADM4; UBA1; SLC6A9; LRRC8A; ATP1B3; SPPL2A; NTRK2; RNF130; LIFR; EMP3; PCDH7; NTRK3; COL6A1; IL17D; LRP10; ADAM19; SGCE; FAT1; SLC44A1; LTBP3; SLC39A10; ABCA1; SYPL1; SLITRK2; GNPTG; CD302; MRC2; LRP4; CALU; CD151; SORL1; TSPAN6; LRRN1; TENM2; CAPNS1; NLGN1; SLC15A2; NLGN4X; EGFR; ADORA1; SLC9A7; SIRPA; EFCAB14; ANGPTL1; FGFR1; VAMP5; CLDN12; LAMB2; GPR155; FGFR2; SLC44A2; LRP1B; PTGFRN; FNDC5; NOTCH1; DPY19L4; S1PR1; CD44; TNFRSF1A; FAM234A; CDH4; HLA-E; COL11A1; NCAM2; AQP1; CPQ; EMP1; FCGRT; GPC5; ROBO1; VCAN; LGALS3BP; LDLR; LRRC4B; NOTCH2; ALCAM; RYR3; SLC9A9; TMEM94; VCAM1; IGSF1; PCDHA10; CDH10; CACHD1; P2RX7; AEBP1; PLXNB1; AXL; ALPL; ST3GAL4; SERPINI1; ITGB5; CD58; FGFRL1; PLPP1; TTYH2; IL17RB; FAM171A1; IL17RD; ANO6; ADAM22; PTPRG; ANTXR1; ZDHHC23; AGRN; COL14A1; POSTN; CNTFR; SEMA5A; FLNA; EMP2; TFPI; ITGA7; MXRA8; TENM4; FSTL1; CD82; NRP2; GPC4; ARSF; LAMC1; KIT; SEMA4A; LTBP1; and CSF1. In certain embodiments, the specific subset of astrocyte markers used in negatively sorting astrocytes from photoreceptor cells is one or more of the following markers: ADGRL4; SERPINE2; BCHE; ABI3BP; NRP1; FSTL1; FAT1; NTRK2; FBLN2; PRSS35; SLC1A3; FCGRT; LAMC1; TF; SORCS2; DKK3; LRP1; PTPRD; ANGPTL1; LTBP3; CLU; CNTNAP2; CD151; PCDH9; CRIM1; CSPG5; and PMEPA1; and the specific subset of BSL cell markers used in negatively sorting BSL cells from photoreceptor cells is one or more of the following markers: HEPACAM; FGFR3; SERPINE2; BCAN; CCDC80; PLPP3; CHL1; ADGRG1; SLC6A11; LSAMP; FBLN2; F3; SLC1A3; DKK3; LRP1; DNER; CLU; PCDH9; and CSPG5.

In certain embodiments, at least about 60% of the cells of the sorted retinal cell populations of the present disclosure express a marker of photoreceptor cell identity. For example, but not by way of limitation, the marker of photoreceptor cell identity is CRX or RCVRN. In certain embodiments, at least about 65% of the cells of the retinal cell populations of the present disclosure express a marker of photoreceptor cell identity. In certain embodiments, at least about 70% of the cells of the retinal cell populations of the present disclosure express a marker of photoreceptor cell identity. In certain embodiments, at least about 75% of the cells of the retinal cell populations of the present disclosure express a marker of photoreceptor cell identity. In certain embodiments, at least about 80% of the cells of the retinal cell populations of the present disclosure express a marker of photoreceptor cell identity. In certain embodiments, at least about 85% of the cells of the retinal cell populations of the present disclosure express a marker of photoreceptor cell identity. In certain embodiments, at least about 90% of the cells of the retinal cell populations of the present disclosure express a marker of photoreceptor cell identity. In certain embodiments, at least about 90% of the cells of the retinal cell populations of the present disclosure express a marker of photoreceptor cell identity. In certain embodiments, at least about 95% of the cells of the retinal cell populations of the present disclosure express a marker of photoreceptor cell identity. In certain embodiments, up to about 100% of the cells of the retinal cell populations of the present disclosure express a marker of photoreceptor cell identity.

In certain embodiments, at least about 15% to about 45% of the cells of the retinal cell populations of the present disclosure express at least one marker of cone photoreceptor cell identity. For example, but not by way of limitation, the marker of cone photoreceptor cell identity can be CNGA3, CNGB3, ARR3, THRB, or S-opsin. In certain embodiments, at least about 20% to about 45% of the cells of the retinal cell populations of the present disclosure express a marker of cone photoreceptor cell identity. In certain embodiments, at least about 25% to about 45% of the cells of the retinal cell populations of the present disclosure express a marker of photoreceptor cell identity. In certain embodiments, at least about 30% to about 45% of the cells of the retinal cell populations of the present disclosure express a marker of cone photoreceptor cell identity. In certain embodiments, at least about 35% to about 45% of the cells of the retinal cell populations of the present disclosure express a marker of cone photoreceptor cell identity. In certain embodiments, at least about 40% to about 45% of the cells of the retinal cell populations of the present disclosure express a marker of cone photoreceptor cell identity.

In certain embodiments, at least about 30% of the cells of the retinal cell populations expressing at least one marker of cone photoreceptor cell identity express CNGA3. In certain embodiments, at least about 30% of the cells of the retinal cell populations expressing at least one marker of cone photoreceptor cell identity express CNGB3. In certain embodiments, at least about 20% of the cells of the retinal cell populations expressing at least one marker of cone photoreceptor cell identity express ARR3.In certain embodiments, at least about 3% of the cells of the retinal cell populations expressing at least one marker of cone photoreceptor cell identity express THRB. In certain embodiments, at least one cell of the retinal cell populations expressing at least one marker of cone photoreceptor cell identity expresses S-opsin.

In certain embodiments, at least about 30% of the cells of the retinal cell populations expressing at least one marker of cone photoreceptor cell identity express CNGA3, at least about 30% of the cells of the retinal cell populations expressing at least one marker of cone photoreceptor cell identity express CNGB3, at least about 20% of the cells of the retinal cell populations expressing at least one marker of cone photoreceptor cell identity express ARR3, at least about 3% of the cells of the retinal cell populations expressing at least one marker of cone photoreceptor cell identity express THRB, and at least one cell of the retinal cell populations expressing at least one marker of cone photoreceptor cell identity expresses S-opsin.

In certain embodiments, at least about 55% to about 85% of the cells of the retinal cell populations of the present disclosure express at least one marker of rod photoreceptor cell identity. For example, but not by way of limitation, the marker of rod photoreceptor cell identity can be NRL, NR2E3, PDE6B, CNGA1, or RHO. In certain embodiments, at least about 60% to about 85% of the cells of the retinal cell populations of the present disclosure express a marker of rod photoreceptor cell identity. In certain embodiments, at least about 65% to about 85% of the cells of the retinal cell populations of the present disclosure express a marker of rod photoreceptor cell identity. In certain embodiments, at least about 70% to about 85% of the cells of the retinal cell populations of the present disclosure express a marker of rod photoreceptor cell identity. In certain embodiments, at least about 75% to about 85% of the cells of the retinal cell populations of the present disclosure express a marker of rod photoreceptor cell identity. In certain embodiments, at least about 80% to about 85% of the cells of the retinal cell populations of the present disclosure express a marker of rod photoreceptor cell identity

In certain embodiments, at least about 50% of the cells of the retinal cell populations expressing at least one marker of rod photoreceptor cell identity express NRL. In certain embodiments, at least about 40% of the cells of the retinal cell populations expressing at least one marker of rod photoreceptor cell identity express NR2E3. In certain embodiments, at least about 20% of the cells of the retinal cell populations expressing at least one marker of rod photoreceptor cell identity express PDE6B. In certain embodiments, at least about 30% of the cells of the retinal cell populations expressing at least one marker of rod photoreceptor cell identity express CNGA1. In certain embodiments, at least one cell of the retinal cell cluster expressing at least one marker of rod photoreceptor cell identity expresses RHO.

In certain embodiments, at least about 50% of the cells of the retinal cell populations expressing at least one marker of rod photoreceptor cell identity express NRL, at least about 40% of the cells of the retinal cell populations expressing at least one marker of rod photoreceptor cell identity express NR2E3, at least about 20% of the cells of the retinal cell populations expressing at least one marker of rod photoreceptor cell identity express PDE6B, at least about 30% of the cells of the retinal cell populations expressing at least one marker of rod photoreceptor cell identity express CNGA1, and at least one cell of the retinal cell populations expressing at least one marker of rod photoreceptor cell identity expresses RHO.

In certain embodiments, the cells of the retinal cell populations of the present disclosure are selected such that they comprise no more than about 40% cells that express a marker of non-photoreceptor cell identity. For example, but not by way of limitation, markers of non-photoreceptor cell identity are those markers associated with: bipolar cells, Muller glia cells, retinal microglia, forebrain neural progenitor cells, retinal progenitor cells, horizontal cells, ganglion cells, retinal amacrine cells, and retinal pigment epithelium cells.

In certain embodiments, the cells of the retinal cell populations of the present disclosure are selected such that they comprise less than about 10% of bipolar cells. In certain embodiments, the marker associated with bipolar cell identity is one or more of ISL1,SEBOX, CAPB5, BHLHE23, GRM6, SCGN, NRN1L, GRIK1, KLHDC8A, and PROX.

In certain embodiments, the cells of the retinal cell populations of the present disclosure are selected such that they comprise less than about 20% Muller glia cells. In certain embodiments, the marker associated with Muller glia cell identity is one or more of AQP4, PRDX6, VIM, HES1, SLC1A3, GLUL, CLU, RLBP1 and LHX2.

In certain embodiments, the cells of the retinal cell populations of the present disclosure are selected such that they comprise less than about 10% retinal microglia cells. In certain embodiments, the marker associated with retinal microglia cell identity is one or more of PTPRC, MPEG1, and CXCR1.

In certain embodiments, the cells of the retinal cell populations of the present disclosure are selected such that they comprise less than about 5% forebrain neural progenitor cells. In certain embodiments, the marker associated with forebrain neural progenitor cell identity is one or more of NKX2.2, RGCC, NEUROD1, BTG2, GADD45A, and GADD45G.

In certain embodiments, the cells of the retinal cell populations of the present disclosure are selected such that they comprise less than about 3% retinal progenitor cells. In certain embodiments, the marker associated with retinal progenitor cell identity is one or more of HOPX, CDK4, CCND2, VSX2, FGF19, SFRP2, CCNB2, and CCND1.

In certain embodiments, the cells of the retinal cell populations of the present disclosure are selected such that they comprise less than about 10% horizontal cells. In certain embodiments, the marker associated with horizontal cell identity is one or more of ONECUT2, ONECUT1, and LHX1.

In certain embodiments, the cells of the retinal cell populations of the present disclosure are selected such that they comprise less than about 10% retinal ganglion cells. In certain embodiments, the marker associated with retinal ganglion cell identity is one or more of POU4F1, THY1, BRN3B, POU4F2, POU4F3, ISL2, RBPMS, and SNCG.

In certain embodiments, the cells of the retinal cell populations of the present disclosure are selected such that they comprise less than about 5% retinal amacrine cells. In certain embodiments, the marker associated with retinal amacrine cell identity is one or more of TFAP2A, TFAP2B, ELAVL3, NeuN, and ELAVL4.

In certain embodiments, the cells of the retinal cell populations of the present disclosure are selected such that they comprise less than about 10% retinal pigment epithelium cells. In certain embodiments, the marker associated with retinal pigment epithelium cell identity is one or more of BEST1, TIMP3, GRAMD3, and PITPNA.

In certain embodiments, the cells of the retinal cell populations of the present disclosure are selected such that less than 30% of the cells express a marker associated with inflammatory cell identity. For example, but not by way of limitation, markers of inflammatory cell identity are: CD15, CD133, A2B5, and CD38. In certain embodiments, the cells of the retinal cell populations of the present disclosure are selected such that they comprise less than about 30% cells expressing A2B5 and/or CD38. In certain embodiments, the cells of the retinal cell populations of the present disclosure are selected such that they comprise no more than one cell expressing CD15 or CD133.

The present disclosure provides a sorted cell population of in vitro differentiated retinal cells, wherein at least about 50% (e.g., at least about 55%, at least about 60%, at least about 65%, at least about 70%, at least about 75%, at least about 80%, at least about 85%, at least about 90%, at least about 95%, or at least about 99%) of the differentiated cells express at least one marker of photoreceptor cell identity.

In certain embodiments, the present disclosure provides a sorted cell population of in vitro differentiated retinal cells, wherein less than at least about 40% (e.g., less than about 35%, less than about 30%, less than about 25%, less than about 20%, less than about 15%, less than about 10%, less than about 5%, less than about 4%, less than about 3%, less than about 2%, less than about 1%, less than about 0.5%, or less than about 0.1%) of the differentiated cells express at least one marker of non-photoreceptor cell identity.

In certain embodiments, the sorted population of in vitro differentiated retinal cells comprises from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ in vitro differentiated photoreceptor cells.

The presently disclosure also provides compositions comprising such sorted populations of in vitro differentiated retinal cells. In certain embodiments, the sorted population of in vitro differentiated retinal cells are obtained by the differentiation methods described herein. In certain embodiments, said composition is frozen. In certain embodiments, said composition further comprises at least one cryoprotectant, for example, but not limited to, dimethylsulfoxide (DMSO), glycerol, polyethylene glycol, sucrose, trehalose, dextrose, or a combination thereof.

In certain embodiments, the composition is a pharmaceutical composition that comprises a pharmaceutically acceptable carrier. The compositions can be used for preventing and/or treating an inherited or acquired retinal degenerative disease, e.g., retinitis pigmentosa, choroideremia, Stargardt disease, cone-rod dystrophy, Leber Congenital Amaurosis and age related macular degeneration, including, but not limited to “dry” age related macular degeneration and “wet” age related macular degeneration.

### 4. Method of Treating Inherited Retinal Degenerative Diseases

The sorted retinal cell populations and compositions disclosed herein can be used for preventing and/or treating inherited and/or acquired retinal degenerative diseases. For example, but not by way of limitation, the sorted retinal cell populations and compositions disclosed herein can be used for CCTT, which, without being bound by theory, is understood to act by repairing the dysfunctional photoreceptor cells present in a recipient's retina. Again, without being bound by theory, it is understood that the sorted retinal cell populations and compositions disclosed herein exert their therapeutic effect, at least in part, by transferring healthy cellular components, e.g., organelles including mitochondria along with other nuclear, cell membrane-bound, and/or cytoplasmic components, e.g., therapeutic proteins. Thus, the presently disclosed subject matter provides for methods of preventing and/or treating inherited and/or acquired retinal degenerative diseases. In certain embodiments, the methods comprise administering the presently disclosed sorted populations of retinal cells, e.g., stem-cell-derived retinal cells, or compositions comprising thereof to a subject suffering from an inherited or acquired retinal degenerative disease. In certain embodiments, the compositions described herein are pharmaceutical compositions further comprising a pharmaceutically acceptable carrier.

CCTT is effective in multiple mutation classes. For example, CCT is effective in X-linked mutations, autosomal dominant (AD) mutations, autosomal recessive (AR) mutations, and non-mendelian, e.g., mitochondrial, mutations. In addition, with respect to AD mutations, CCTT is effective in haploinsufficiency or dominant negative mutations (e.g., dominant negative interference mutations and dominant negative toxicity mutations). CCTT has also been shown effective in transferring multiple types of cellular components, e.g., membrane-bound proteins, nuclear-localized proteins, cytoplasmic proteins. CCTT is also effective in transferring cellular components to both types of photoreceptor cells, i.e., both rods and cones.

Non-limiting examples of inherited retinal degenerative diseases include retinitis pigmentosa, choroideremia, Stargardt disease, cone-rod dystrophy, and Leber Congenital Amaurosis. Non-limiting examples of acquired retinal degenerative diseases include, age related macular degeneration, including, but not limited to “dry” age related macular degeneration and “wet” age related macular degeneration.

The sorted populations of retinal cells or compositions described herein can be administered in any physiologically acceptable vehicle. The cells or compositions of the present disclosure can be administered via localized injection or via subretinal transplant. In certain embodiments, the sorted populations of cells or compositions will be resuspended in media and transplanted into the subretinal space using a device that preserves their biologic activity and ensures on-target placement. In certain embodiments, the device will be comprised of biocompatible materials. In certain embodiments, the device will accomplish the transplant with limited shear stress on cells, e.g., it will comprise a low-friction passage. An exemplary device for subretinal transplant is described in International Patent Application No. PCT/US2019/045074 (Published as WO2020028892), which is incorporated herein by reference in its entirety.

The cells or compositions described herein can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, e.g., a composition comprising the presently disclosed stem-cell-derived retinal cells, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin.

Viscosity of the compositions, if desired, can be maintained at the selected level using a pharmaceutically acceptable thickening agent. Methylcellulose can be used because it is readily and economically available and is easy to work with. Other suitable thickening agents include, for example, hyaluronic acid, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the non-cellular derived components of the compositions should generally, but not exclusively, be selected to be chemically inert and thus not affect the viability or efficacy of the presently disclosed retinal cells. This will present no problem to those skilled in chemical and pharmaceutical principles, or problems can be readily avoided by reference to standard texts or by simple experiments (not involving undue experimentation), from this disclosure and the documents cited herein.

In certain embodiments, the compositions described herein comprise an effective amount of the sorted retinal cells. As used herein, the term “effective amount” or “therapeutically effective amount” refers to an amount sufficient to affect a beneficial or desired clinical result upon treatment. An effective amount can be administered to a subject in at least one dose. In terms of treatment, an effective amount is an amount that is sufficient to palliate, ameliorate, stabilize, reverse or slow the progression of the inherited or acquired retinal degenerative disease, or otherwise reduce the pathological consequences of the inherited or acquired retinal degenerative disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

In certain embodiments, an effective amount of the cells is an amount that is sufficient to improve the retinal function of a subject suffering from an inherited or acquired retinal degenerative disease. In certain embodiments, an effective amount of the cells is an amount that is sufficient to improve the retinal function of a subject suffering from an inherited or acquired retinal degenerative disease, e.g., the improved function can be about 1%, about 5%, about 10%, about 20%, about 30%, about 40%, about 50%, about 60%, about 70%, about 80%, about 90%, about 95%, about 98%, about 99% or about 100% of the retinal function of an individual not suffering from the inherited or acquired retinal degenerative disease.

The quantity of sorted cells to be administered will vary for the subject being treated. In certain embodiments, from about 1×10⁴ to about 1×10¹⁰, from about 1×10⁴to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ of the sorted cells are administered to a subject. In certain embodiments, from about 1×10⁵ to about 1×10⁷ of the sorted cells are administered to a subject suffering from an inherited or acquired retinal degenerative disease. In certain embodiments, from about 1×10⁶ to about 1×10⁷ of the sorted cells are administered to a subject suffering from an inherited or acquired retinal degenerative disease. In certain embodiments, from about 1×10⁶ to about 4×10⁶ of the sorted cells are administered to a subject suffering from an inherited or acquired retinal degenerative disease. The precise determination of what would be considered an effective dose may be based on factors individual to each subject, including their size, age, sex, weight, and condition of the particular subject. Dosages can be readily ascertained by those skilled in the art from this disclosure and the knowledge in the art.

### EXEMPLARY EMBODIMENTS

A. In certain non-limiting embodiments, the presently disclosed subject matter provides for an in vitro method to produce sorted population of retinal cells, comprising: generating a three-dimensional retinal organoid; dissociating the three-dimensional retinal organoid; and positively sorting retinal cells based on one or more marker of photoreceptor cell identity and/or negatively sorting retinal cells based on one or more marker of non-photoreceptor cell identity to produce the sorted population of retinal cells.

A1. The foregoing method of A, wherein: the marker of photoreceptor cell identity is CD73; the marker of non-photoceptor cell identity is one or more of CD24, CD302, CD9, and CD99; the marker of non-photoreceptor cell identity is one or more of ITM2B; CD63; ENO1; CALR; CANX; CLU; SLC3A2; BSG; GPM6B; ITGB1; PTTG1IP; TIMP1; PMEPA1; SSR2; DKK3; LRP1; ATRAID; HLA-A; HLA-C; EMP3; TMED9; GOLIM4; LTBP3; GALNT1; CD151; PLD3; CALU; LSAMP; CD59; SLC2A1; LAMP2; HLA-B; COL11A1; DPP7; DCBLD2; CD164; SLC1A3; F3; CTSD; FLNA; SLC39A10; FN1; TMEM106C; TMEM179B; ATP1B3; HLA-E; TMEM132A; FLT1; FGFR1; CAPNS1; FAT1; ANGPTL1; LRP10; CRELD2; SPPL2A; TSPAN4; PRSS35; ECE1; SYPL1; SORCS2; COL2A1; DNER; COL6A1; CD44; GPC1; PCDH9; CRIM1; CHL1; TTYH3; IKBIP; NECTIN2; FBLN2; CCDC80; DAG1; PBXIP1; PRSS23; ACAA1; NTRK2; FSTL1; BCHE; TNFRSF1A; LGALS3BP; ITGB8; CP; ADGRG1; VCAN; OLFM1; NRP1; SCARB1; TSPAN6; PCDH17; PLTP; NECTIN3; PTPRD; CADM4; UNC5B; CSPG5; AXL; PLXNB2; PLPP3; NOTCH2; SLITRK2; AEBP1; ANGPTL4; COLEC12; VAMP5; NLGN4X; FGFRL1; EFNB2; COL5A1; LAMB2; LAMC1; IGFBP3; FNDC5; FCGRT; ADRA2C; SERPING1; EPHB2; CDH11; COL1A2; CNTFR; AGRN; ROBO1; LOX; MRC2; COL6A2; SLC6A11; DSC2; IGSF8; EMP1; ABI3BP; TTYH2; NOTCH1; ANO6; A2M; SORCS1; EFNA1; PTPRG; TF; EMP2; CEMIP2; SERPINE2; CDON; EGFR; PCDH7; MAN2A1; IL1R1; COL1A1; SEMA5A; ANTXR1; SIPR3; ITPRIP; MXRA8; PRELP; AQP1; CSF1; BCAN; ADGRA2; CA12; FAT3; HEPACAM; FGFR3; TRIL; HSD17B2; and HP.

A2. The foregoing method of A, wherein the marker of non-photoreceptor cell identity is one or more of DKK3; LRP1; CLU; PMEPA1; ITGB1; and PTTG1IP.

A3. The foregoing method of A, wherein the one or more marker of non-photoreceptor cell identity is one or more astrocyte marker selected from ITM2B; CD63; ENO1; CALR; CANX; CLU; SLC3A2; BSG; GPM6B; ITGB1; PTTG1IP; TIMP1; PMEPA1; SSR2; DKK3; LRP1; ATRAID; HLA-A; HLA-C; EMP3; TMED9; GOLIM4; LTBP3; GALNT1; CD151; PLD3; CALU; LSAMP; CD59; SLC2A1; LAMP2; HLA-B; COL11A1; DPP7; DCBLD2; CD164; SLC1A3; F3; CTSD; FLNA; SLC39A10; FN1; TMEM106C; TMEM179B; ATP1B3; HLA-E; TMEM132A; FLT1; FGFR1; CAPNS1; FAT1; ANGPTL1; LRP10; CRELD2; SPPL2A; TSPAN4; PRSS35; ECE1; SYPL1; SORCS2; COL2A1; DNER; COL6A1; CD44; GPC1; PCDH9; CRIM1; CHL1; TTYH3; IKBIP; NECTIN2; FBLN2; CCDC80; DAG1; PBXIP1; PRSS23; ACAA1; NTRK2; FSTL1; BCHE; TNFRSF1A; LGALS3BP; ITGB8; CP; ADGRG1; VCAN; OLFM1; NRP1; SCARB1; TSPAN6; PCDH17; PLTP; NECTIN3; PTPRD; CADM4; UNC5B; CSPG5; AXL; PLXNB2; PLPP3; NOTCH2; SLITRK2; AEBP1; ANGPTL4; COLEC12; VAMP5; NLGN4X; FGFRL1; EFNB2; COL5A1; LAMB2; LAMC1; IGFBP3; FNDC5; FCGRT; ADRA2C; SERPING1; EPHB2; CDH11; COL1A2; CNTFR; AGRN; ROBO1; LOX; MRC2; COL6A2; SLC6A11; DSC2; IGSF8; EMP1; ABI3BP; TTYH2; NOTCH1; ANO6; A2M; SORCS1; EFNA1; PTPRG; TF; EMP2; CEMIP2; SERPINE2; CDON; EGFR; PCDH7; MAN2A1; IL1R1; COL1A1; SEMA5A; ANTXR1; SIPR3; ITPRIP; MXRA8; PRELP; AQP1; CSF1; BCAN; ADGRA2; CA12; FAT3; HEPACAM; FGFR3; TRIL; HSD17B2; and HP.

A4. The foregoing method of A3, wherein the one or more marker of non-photoreceptor cell identity is one or more astrocyte marker selected from ADGRL4; SERPINE2; BCHE; ABI3BP; NRP1; FSTL1; FAT1; NTRK2; FBLN2; PRSS35; SLC1A3; FCGRT; LAMC1; TF; SORCS2; DKK3; LRP1; PTPRD; ANGPTL1; LTBP3; CLU; CNTNAP2; CD151; PCDH9; CRIM1; CSPG5; and PMEPA1.

A5. The foregoing method of A, wherein the one or more marker of non-photoreceptor cell identity is one or more brain and spinal cord-like (BSL) cell marker selected from CLU; ITM2B; PTPRZ1; GPM6B; ATP1B2; CD63; BCAN; SLC1A3; SERPINE2; LRP1; PTPRA; ADGRG1; ENO1; CANX; SLC3A2; DNER; PTTG1IP; CALR; PCDH9; CCDC80; LSAMP; HEPACAM; F3; PLPP3; APLP2; FBLN2; TIMP1; SLC6A11; CSPG5; JAM2; FGFR3; DKK3; GOLIM4; NCAM1; CHL1; NRCAM; HLA-A; TMEM132A; PMEPA1; ITGAV; SSR2; ACAA1; BCHE; CD59; FAT3; PCDH17; ST3GAL5; PBXIP1; LAMP1; ITGB1; HP; ITGB8; SGCB; LAMP2; CLDND1; TMEM106B; PTCH1; PLTP; RNF13; HLA-C; PTPRD; TMEM30A; TRIL; RAB5C; TTYH3; DAG1; CADM4; UBA1; SLC6A9; LRRC8A; ATP1B3; SPPL2A; NTRK2; RNF130; LIFR; EMP3; PCDH7; NTRK3; COL6A1; IL17D; LRP10; ADAM19; SGCE; FAT1; SLC44A1; LTBP3; SLC39A10; ABCA1; SYPL1; SLITRK2; GNPTG; CD302; MRC2; LRP4; CALU; CD151; SORL1; TSPAN6; LRRN1; TENM2; CAPNS1; NLGN1; SLC15A2; NLGN4X; EGFR; ADORA1; SLC9A7; SIRPA; EFCAB14; ANGPTL1; FGFR1; VAMP5; CLDN12; LAMB2; GPR155; FGFR2; SLC44A2; LRP1B; PTGFRN; FNDC5; NOTCH1; DPY19L4; S1PR1; CD44; TNFRSF1A; FAM234A; CDH4; HLA-E; COL11A1; NCAM2; AQP1; CPQ; EMP1; FCGRT; GPC5; ROBO1; VCAN; LGALS3BP; LDLR; LRRC4B; NOTCH2; ALCAM; RYR3; SLC9A9; TMEM94; VCAM1; IGSF1; PCDHA10; CDH10; CACHD1; P2RX7; AEBP1; PLXNB1; AXL; ALPL; ST3GAL4; SERPINI1; ITGB5; CD58; FGFRL1; PLPP1; TTYH2; IL17RB; FAM171A1; IL17RD; ANO6; ADAM22; PTPRG; ANTXR1; ZDHHC23; AGRN; COL14A1; POSTN; CNTFR; SEMA5A; FLNA; EMP2; TFPI; ITGA7; MXRA8; TENM4; FSTL1; CD82; NRP2; GPC4; ARSF; LAMC1; KIT; SEMA4A; LTBP1; and CSF1.

A6. The foregoing method of A5, wherein the one or more marker of non-photoreceptor cell identity is one or more BSL marker selected from HEPACAM; FGFR3; SERPINE2; BCAN; CCDC80; PLPP3; CHL1; ADGRG1; SLC6A11; LSAMP; FBLN2; F3; SLC1A3; DKK3; LRP1; DNER; CLU; PCDH9; and CSPG5.

A7. The foregoing method of A-A1, wherein the three-dimensional retinal organoid is enzymatically dissociated.

A8. The foregoing method of A7, wherein the enzyme is papain and/or trypsin.

A9. The foregoing method of A7, wherein the retinal cells are contacted with a composition to ensure that the cells remain in a dissociated cell suspension.

A10. The foregoing method of A9, wherein the composition is an enzyme.

A11. The foregoing method of A10, wherein the enzyme is DNAse.

A12. The foregoing method of A-A1, wherein the three-dimensional retinal organoid reaches between about DD 45 and DD 300 prior to being dissociated.

A13. The foregoing method of A12, wherein the three-dimensional retinal organoid reaches about DD 90 to about DD 140 prior to being dissociated.

A14. The foregoing method of A-A13, wherein the retinal cell population consists of at least about 70% single cells.

A15. The foregoing method of A14, the retinal cell population consists of at least about 80% single cells.

A16. The foregoing method of A14, wherein the retinal cell population consists of at least about 90% single cells.

A17. The foregoing method of A-A16, wherein the retinal cell population comprises about 55% to about 85% rod photoreceptor cells.

A18. The foregoing method of A-A17, wherein the stem cells are selected from human, nonhuman primate or rodent nonembryonic stem cells; human, nonhuman primate or rodent embryonic stem cells; human, nonhuman primate or rodent induced pluripotent stem cells; and human, nonhuman primate or rodent recombinant pluripotent cells.

A19. The foregoing method of A-A18, wherein stem cells are human stem cells.

A20. The foregoing method of A-A19, wherein the stem cells are pluripotent or multipotent stem cells.

A21. The foregoing method of A-A20, wherein the stem cells are pluripotent stem cells.

A22. The foregoing method of A-A21, wherein the pluripotent stem cells are selected from embryonic stem cells, induced pluripotent stem cells, and combinations thereof.

B. In certain non-limiting embodiments, the presently disclosed subject matter provides for a sorted population of in vitro differentiated retinal cells, wherein said in vitro differentiated retinal cells are obtained by a method of A-A22.

C. In certain non-limiting embodiments, the presently disclosed subject matter provides for a composition comprising the cell population of B.

C1. The foregoing composition of C, which is a pharmaceutical composition further comprising a pharmaceutically acceptable carrier.

D. In certain non-limiting embodiments, the presently disclosed subject matter provides for a method of preventing and/or treating an inherited or acquired retinal degenerative disease in a subject, comprising administering to the subject an effective amount of one of the following: (a) the sorted population of in vitro differentiated retinal cells of B; or (b) the composition of C-C1.

D1. The foregoing method of D, wherein the inherited retinal degenerative disease is selected from retinitis pigmentosa, choroideremia, Stargardt disease, cone-rod dystrophy, and Leber Congenital Amaurosis.

D2. The foregoing method of D, wherein the acquired retinal degenerative disease is age-related macular degeneration.

E. In certain non-limiting embodiments, the presently disclosed subject matter provides for the sorted population of in vitro differentiated retinal cells of claim A18 or the composition of C-C1 for use in preventing and/or treating an inherited or acquired retinal degenerative disease in a subject.

E1. The foregoing sorted population of in vitro differentiated retinal cells or composition for use in preventing and/or treating an inherited retinal degenerative disease in a subject of E, wherein the inherited retinal degenerative disease is retinitis pigmentosa, choroideremia, Stargardt disease, cone-rod dystrophy, or Leber Congenital Amaurosis.

E2. The foregoing sorted population of in vitro differentiated retinal cells or composition for use in preventing and/or treating an acquired retinal degenerative disease in a subject of claim E, wherein the acquired retinal degenerative disease is age-related macular degeneration.

### EXAMPLES

### 1. Human Donor Cells Migrate from or Remain in the Subretinal Space

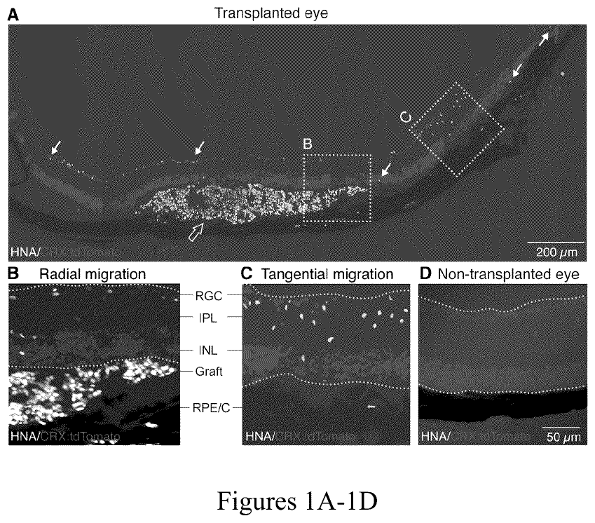

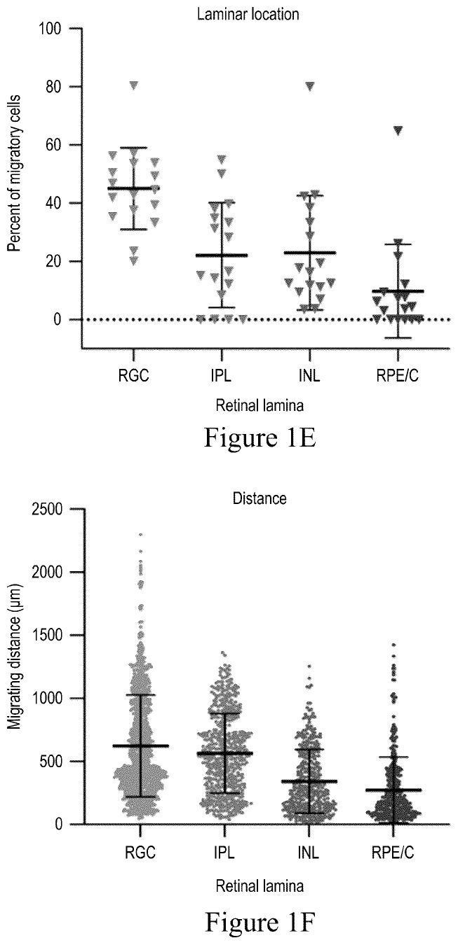

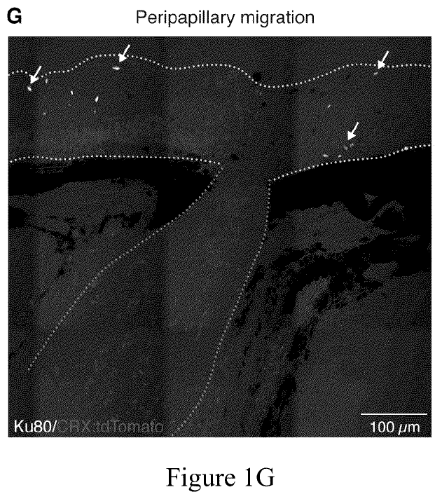

To determine how the recipient subretinal space affects donor cells, we differentiated human retinal organoids, transplanted them into recipient mice, and later assessed donor cell position, fate, and maturity. To generate recipient mice, we crossed and bred mice with immune deficiency and retinal degeneration. These C3H/HeJ-Pde6bR1l/R1(Rd1) and NOD.CB17-Prkdcscid/J (NOD/Scid) double mutant mice are termed Rd1/NS. To generate donor cells, we used H9 human embryonic stem cells (hESCs) carrying a reporter that is expressed in all photoreceptors (CRX:tdTomato). We used a gravity aggregation approach to differentiate stem cells into retinal organoids with robust generation of photoreceptors. On day 134 of organoid culture, we micro-dissected the human retinal organoids and transplanted the fragments into the subretinal space of recipient eyes (n=16 eyes). Four and a half months later, we evaluated the transplants. As homozygosity for the Rd1 allele causes virtually all photoreceptors to degenerate by adulthood in mice, distinct recipient outer nuclear and outer plexiform layers were not observed but the inner nuclear, inner plexiform, retinal ganglion cell (RGC), and retinal nerve fiber layers (collectively, the “inner retina”) were present. We determined the positions of donor cells relative to the subretinal transplantation site. We identified all human donor cells based on immunolabeling for human nuclear antigen (HNA), or human ATP-dependent DNA helicase 2 subunit (Ku80 protein). We identified human donor photoreceptors based on transgenic expression of CRX:tdTomato. We observed two main classes of donor cells: (1) human cells in the recipient subretinal space (“nonmigratory cells”) that were photoreceptor or non-photoreceptor cells; (FIG. 1A); and (2) human cells in the recipient inner retina (“migratory cells”) that were not photoreceptors (FIG. 1A), suggesting that this population had migrated from the graft. Migratory cells traveled into all retinal layers, including the RGC, the inner plexiform layer (IPL), the inner nuclear layer (INL), and the retinal pigment epithelium/choroid (RPE/C) layer (FIG. 1E). A subset of migratory cells were observed in the recipient inner retinal layers overlying the graft (“radial migration”), whereas others migrated tangentially beyond the edges of the graft (“tangential migration”), including the regions flanking the optic nerve (“peripapillary migration” FIG. 1B). 98.9% of tangentially migratory cells (n=2,378 cells) were within 1500 μm of the edges of the graft. The remaining 1.1% traveled beyond 1500 μm and were located exclusively in the retinal ganglion cell (RGC) layer (FIG. 1D). We next sought to molecularly classify the fates of these nonmigratory and migratory cells.

### 2. Donor Cells Adopt Retinal-Derived and Non-Retinal-Derived Cell Fates

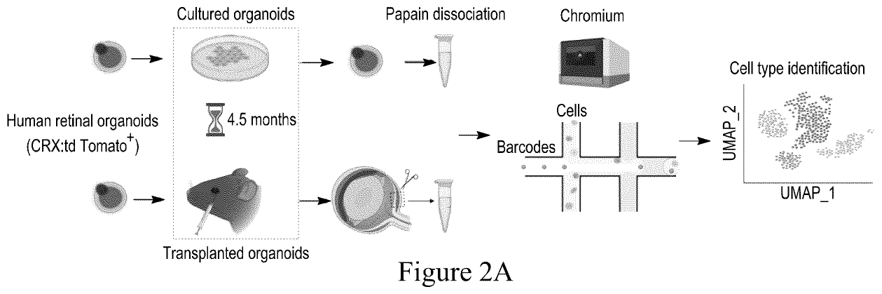

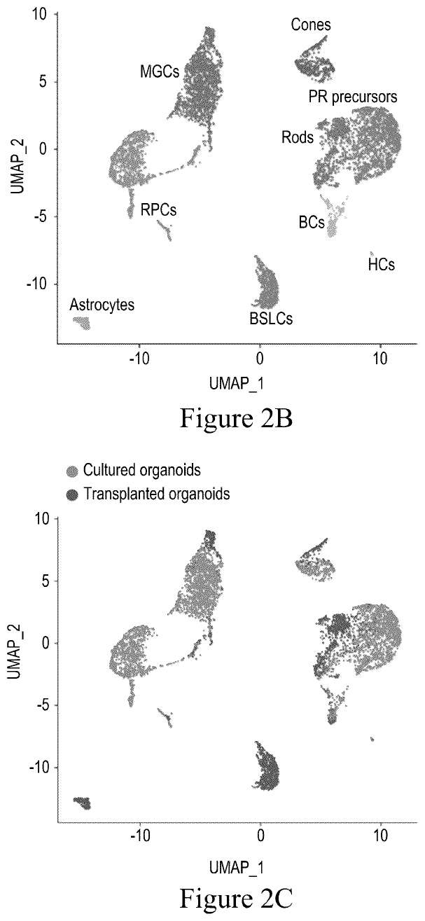

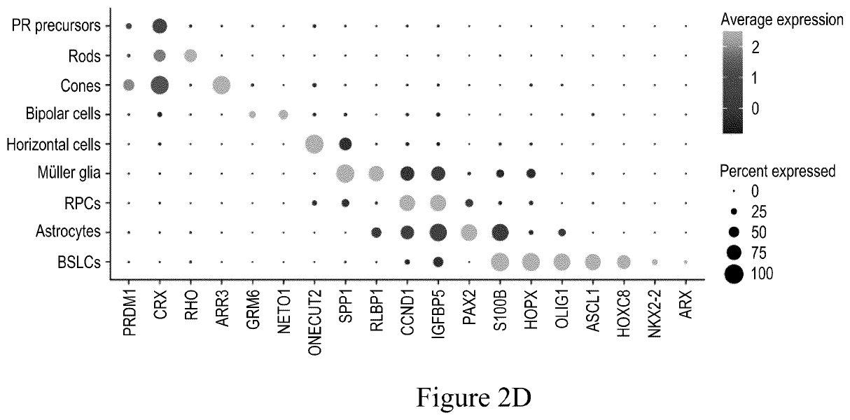

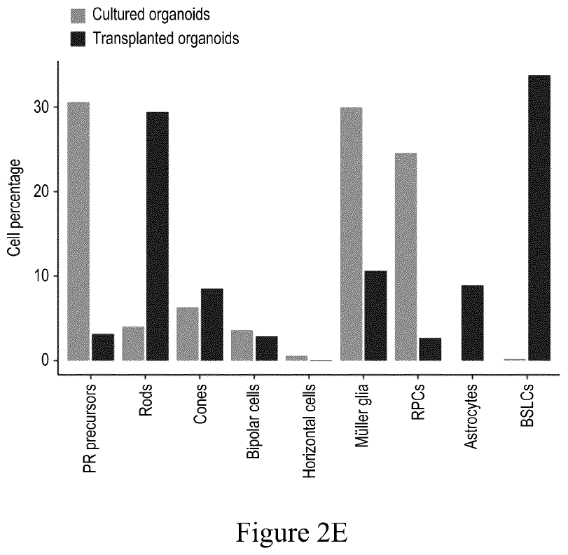

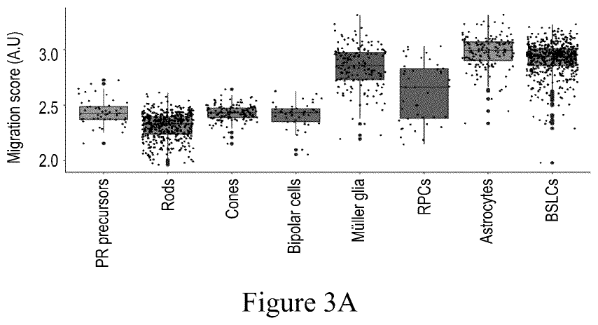

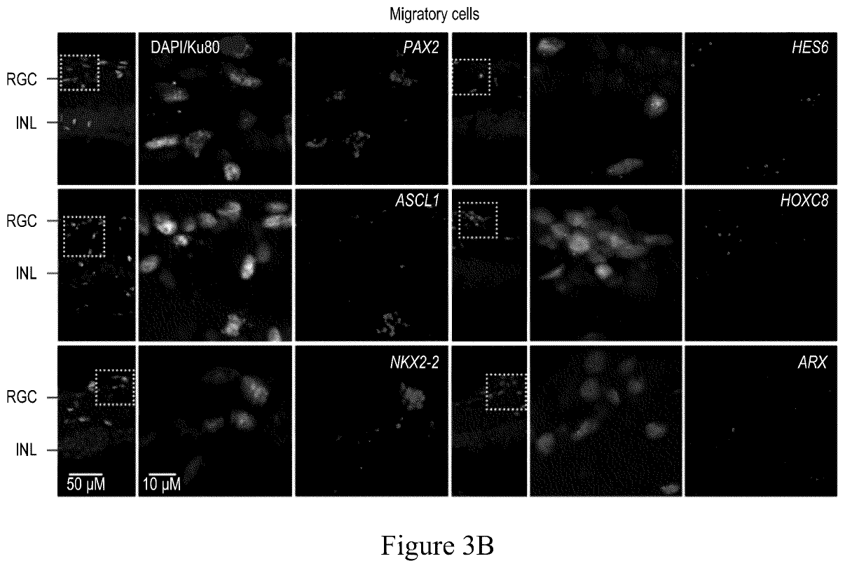

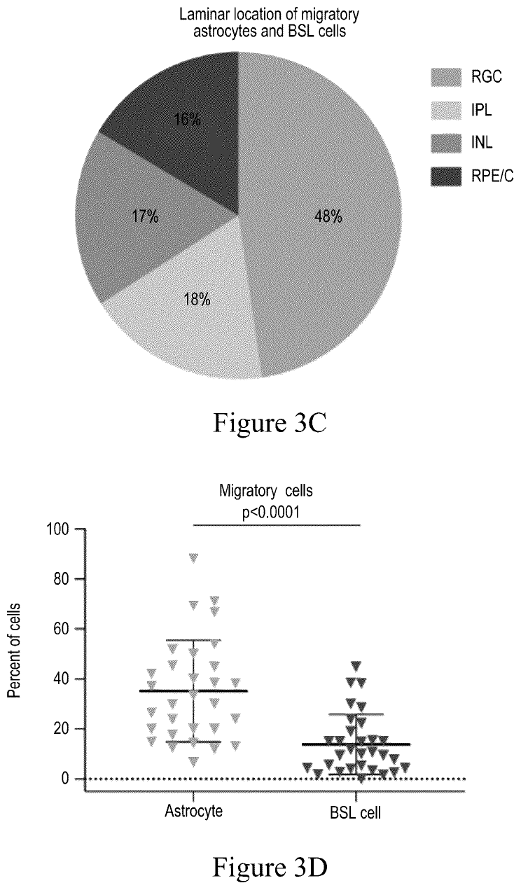

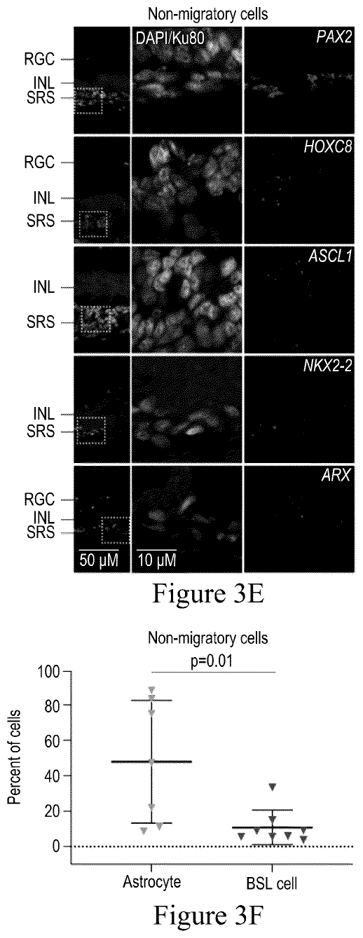

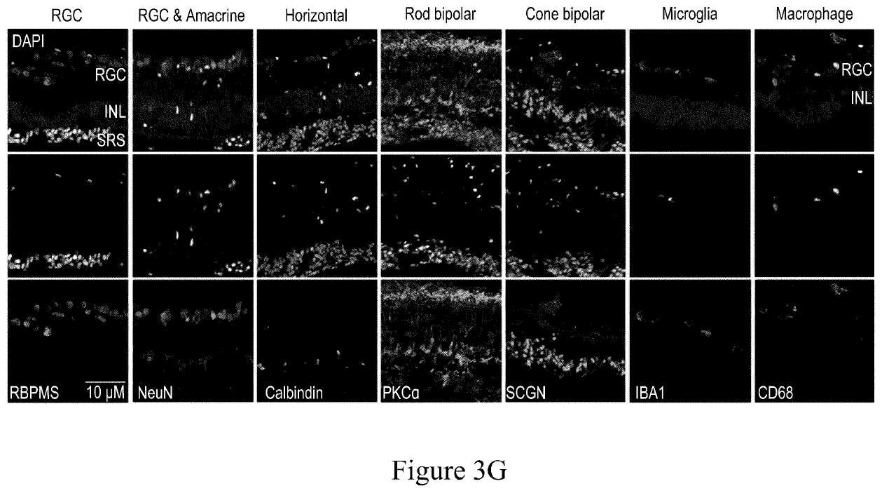

To determine how the recipient subretinal microenvironment affects the gene expression and cell fate specification of the migratory and non-migratory donor cells, we conducted single cell RNA sequencing on cells from human retinal organoids transplanted and matured in vivo (“transplanted organoids”) and from age-matched organoids that were maintained in vitro (“cultured organoids”) (FIG. 2A). We analyzed a total of 5,831 human cells that were recovered from the transplanted (1,561 cells) and cultured (4,270 cells) organoids. We identified retinal cell types including retinal progenitor cells (RPCs), photoreceptor precursor cells, rods, cones, bipolar cells, horizontal cells, and Müller glia based on their gene expression profiles (FIG. 2B-D). The quantities of cones, bipolar cells, and horizontal cells were similar in the transplanted and cultured organoids. In contrast, retinal progenitor cells (RPCs), photoreceptor precursor cells, and Müller glia were more abundant in cultured organoids, whereas rods were more abundant in transplanted organoids (FIG. 2E). The smaller populations of RPCs and photoreceptor precursor cells and larger population of rods in the transplanted organoids suggest that the recipient microenvironment promotes specification and maturation of retinal cell fates.

In addition to these cell types, we identified two cell clusters that could not be ascribed solely to known retinal-derived cell fates. The cells in one cluster expressed genes that are broadly expressed in retinal and other CNS progenitors such as ASCL1 and HES6 (FIG. 2D, Supplementary FIG. S4). They also expressed genes that are not normally detected in the developing retina including NKX2-2 and ARX, both of which are prominently expressed in ventral telencephalic and diencephalic neural progenitors, as well as HOXC8, whose expression is normally restricted to the developing spinal cord (FIG. 2D). Based on this gene expression profile, we designated the cells in this cluster as “brain and spinal cord-like” (BSL) cells. BSL cells comprised approximately 1% of cells in the cultured organoids but were over 30 times more abundant in the transplanted organoids (FIG. 2E). Cells in the second cluster expressed markers characteristic of retinal astrocytes, such as PAX2 and S100B (FIG. 2D). Normally, retinal astrocytes are born in the optic nerve head and migrate into the retina. Strikingly, astrocytes were entirely absent in the cultured organoids, but comprised approximately 8% of cells in the transplanted organoids (FIG. 2E). These data suggest that the recipient microenvironment directs some donor cells to assume fates that are not normally acquired by retinal progenitors.

### 3. Actively Proliferating Cells are Rare Among Migratory and Non-Migratory Donor Cells

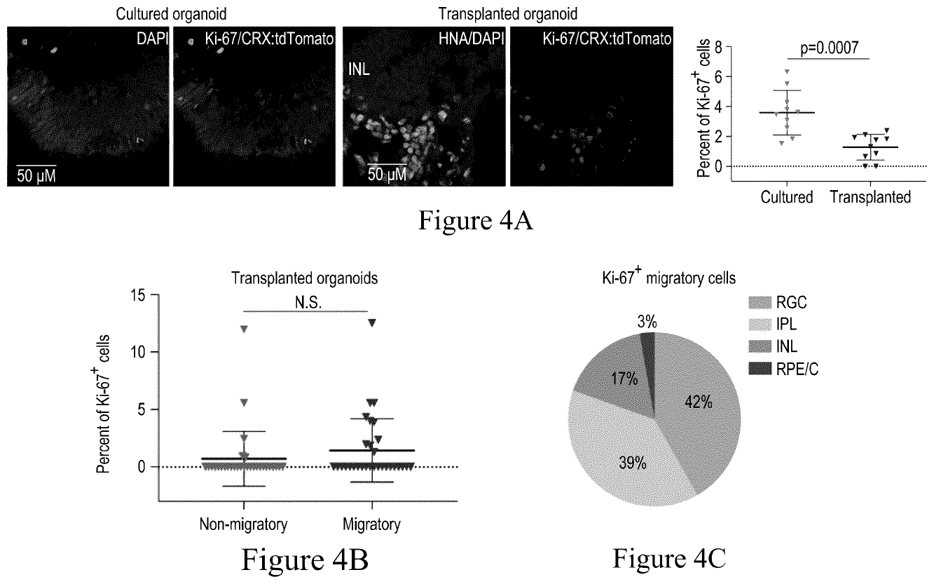

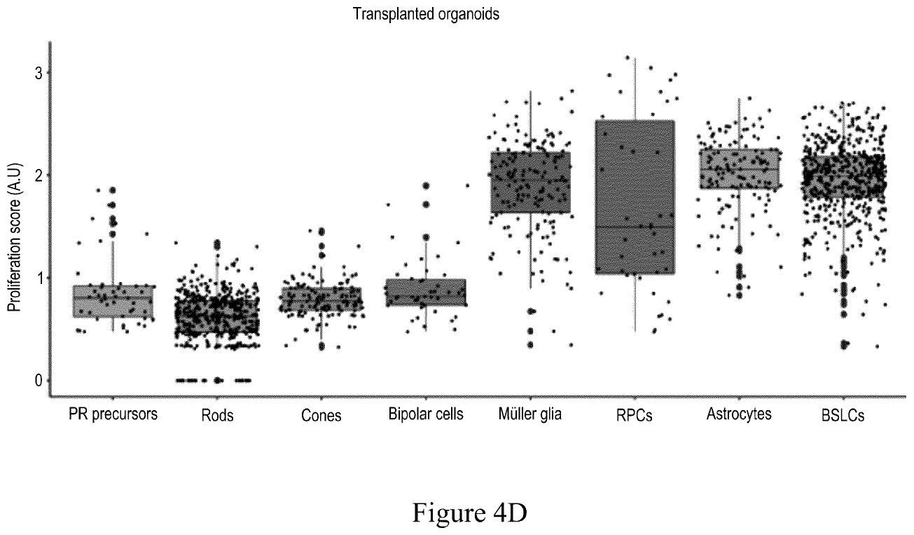

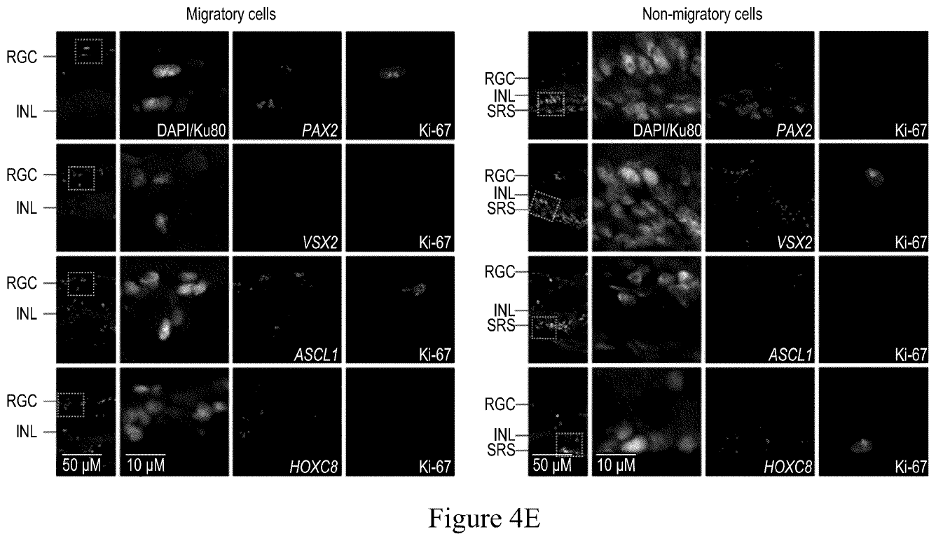

Migratory cells, especially if they are proliferative, may negatively impact the recipient. To determine the influence of the recipient microenvironment on the proliferation of migratory and non-migratory donor cells, we examined expression of the proliferation marker protein Ki-67. As expected, expression of Ki-67 was rarely observed in CRX:tdTomato⁺ photoreceptor precursors in cultured organoids, and were significantly less abundant in transplanted organoids (FIG. 4A). In eyes with transplanted organoids, 0.7% of non-migratory cells and 1.4% of migratory cells expressed Ki-67 (FIG. 4B), and the difference between those values was not statistically significant. We observed that the few Ki-67⁺ migratory cells occupied all retinal laminae of the recipient (FIG. 4C).

To identify the proliferating cells, we developed a proliferation scoring system by computationally aggregating the expression level of proliferation-associated genes (Supplementary data file S2). We found that astrocytes, Müller glia, RPCs, and BSL cells showed the highest proliferation score (FIG. 4D), suggesting that these cells were proliferating. To test this hypothesis, we examined expression of Ki-67 in PAX2+ (astrocytes), VSX2⁺ (RPCs), and ASCL1⁺ and HOXC8⁺ (BSL cells) cells. Precise quantification was impractical due to the rarity of double-positive cells. Nevertheless, we found a few migratory PAX2⁺ astrocytes, and very few migratory BSLs, that were Ki-67+. Proliferating Ki-67⁺/VSX2⁺ RPCs remained in the subretinal space (FIG. 4E).

Taken together, these data suggest that migratory proliferating donor human cells are rare and are mostly astrocytes, and that nonmigratory proliferating cells are rare and are mostly RPCs.

### 4. Donor Cones and Rods Mature More Rapidly in the Recipient Subretinal Space than in Culture

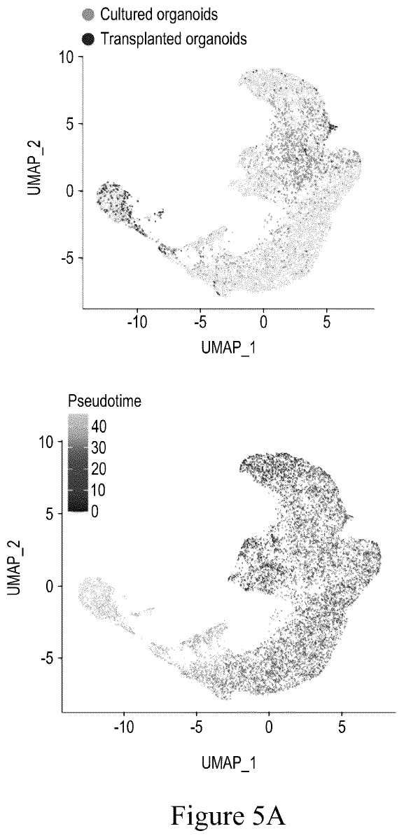

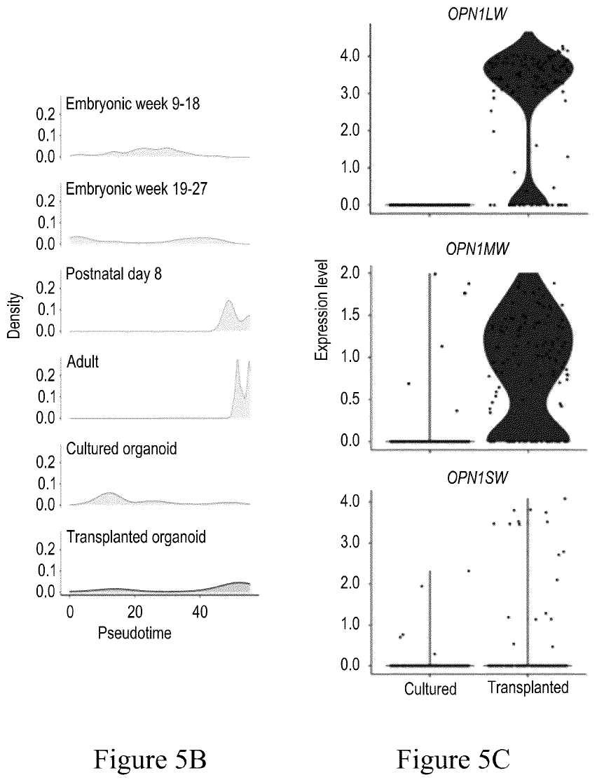

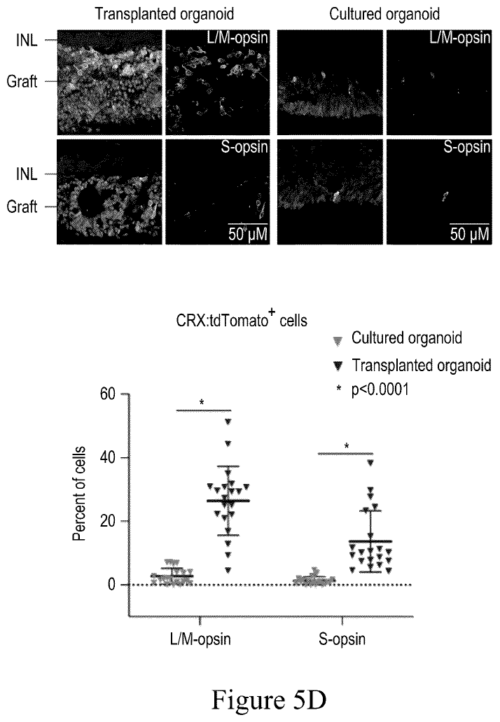

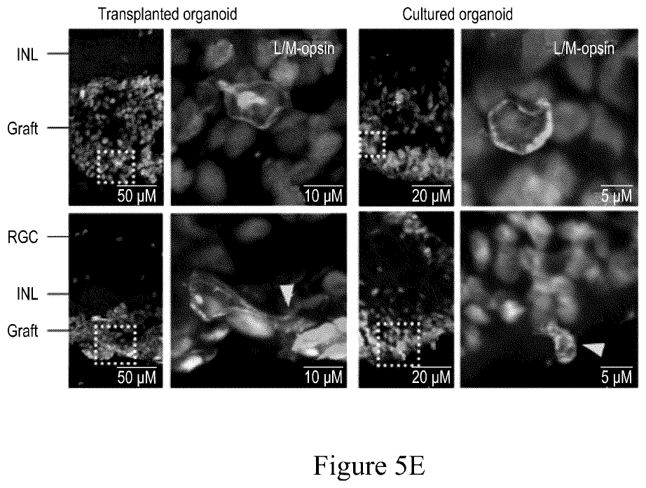

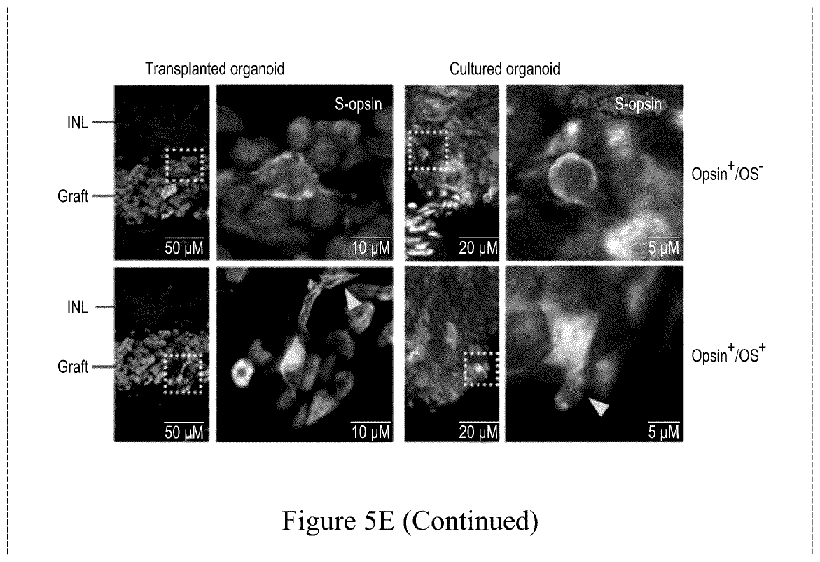

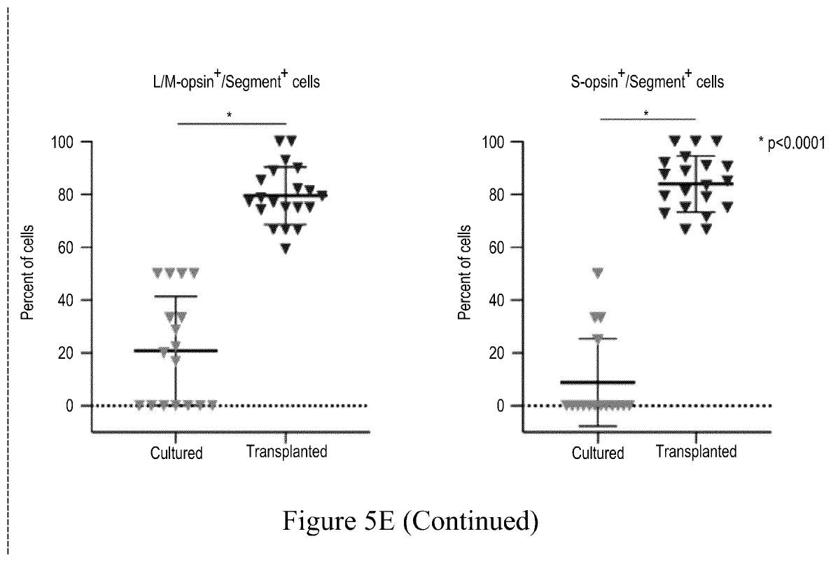

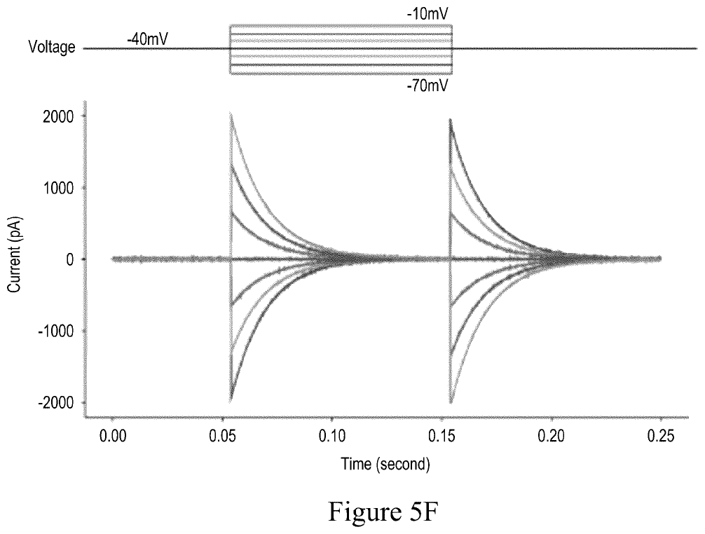

Our scRNA-seq analysis suggested that the recipient subretinal space promotes photoreceptor fate and possibly maturation (FIG. 2D). To test this hypothesis, we first assessed cone maturation. We evaluated the gene expression profiles of cones from transplanted organoids and cultured organoids using pseudotime analysis, comparing these cells to published datasets of embryonic, postnatal, and adult cones isolated directly from human retina (42). The transcriptional profiles suggested that the cones from transplanted organoids resembled adult cones, whereas the cones from cultured organoids more closely resembled embryonic cones (FIG. 5A-B). Expression of mature cone-specific genes were consistently higher in transplanted than in cultured cones (Supplementary FIG. S5), including all three cone opsins (OPN1LW, OPN1MW and OPN1SW) (FIG. 5C). The proportions of CRX:tdTomato⁺ cells that expressed L/M opsin or S opsin were significantly higher in transplanted organoids (L/M-opsin⁺: 26.4%, S-opsin⁺: 28.7%) compared to cultured organoids (L/M-opsin⁺: 2.7%, S-opsin⁺: 1.3%) (FIG. 5D). Similarly, the fraction of L/M opsin⁺ or S opsin cells⁺ with inner or outer segments was significantly higher in transplanted organoids than cultured organoids (FIG. 5E). We measured the intrinsic electrical properties of a transplanted human cone cell and found large capacitance currents (˜2 nA), indicating relatively large cell membrane areas as normally observed in mature cones (FIG. 5F).

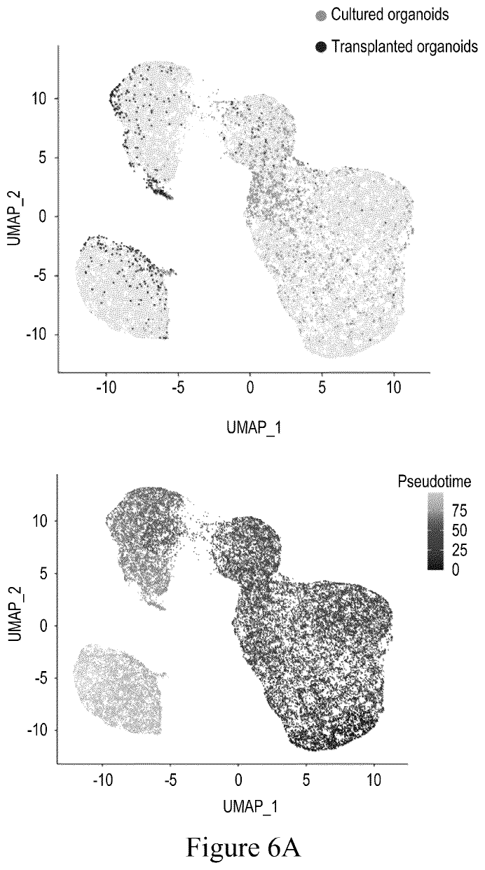

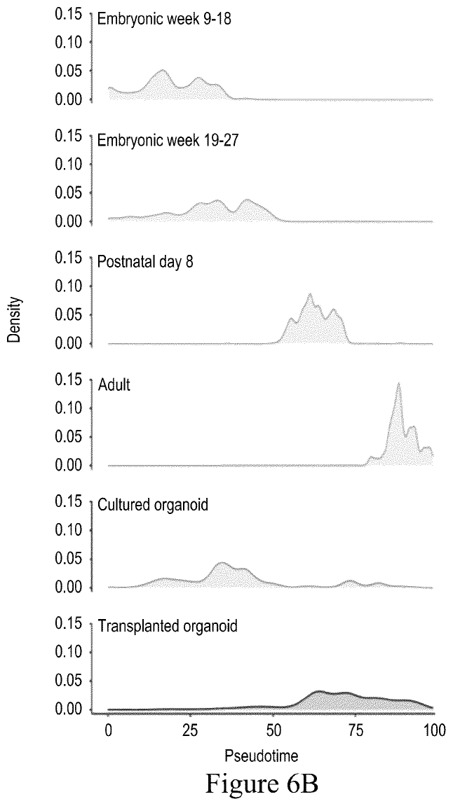

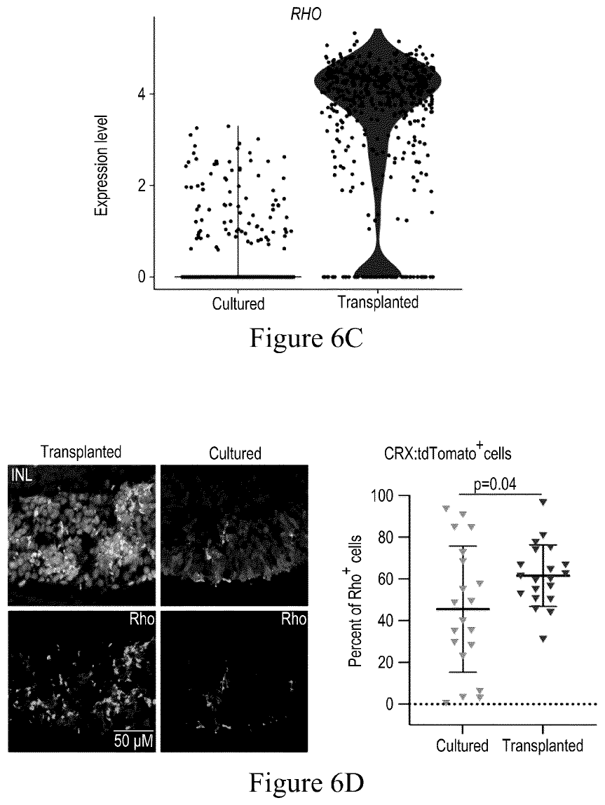

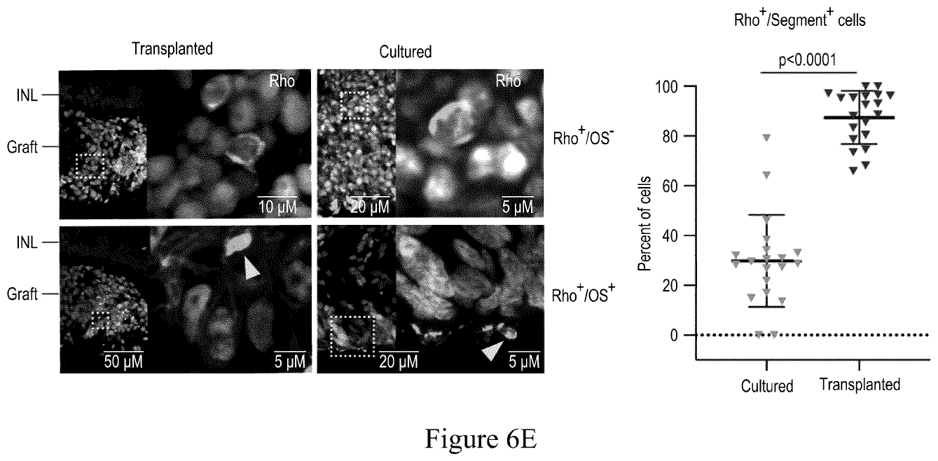

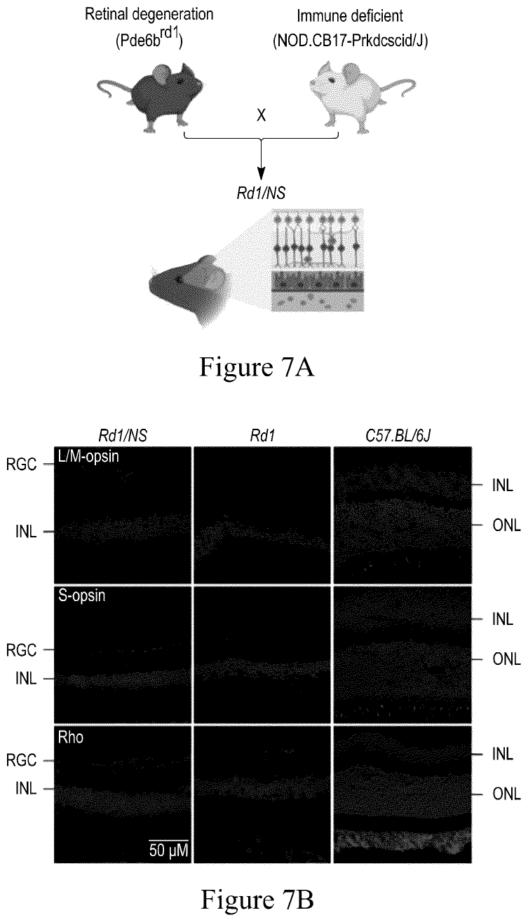

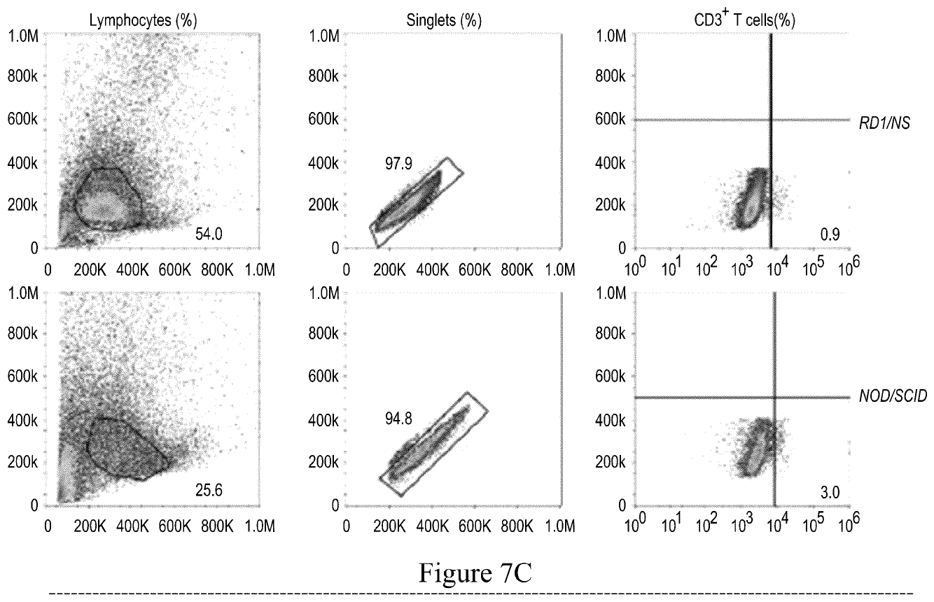

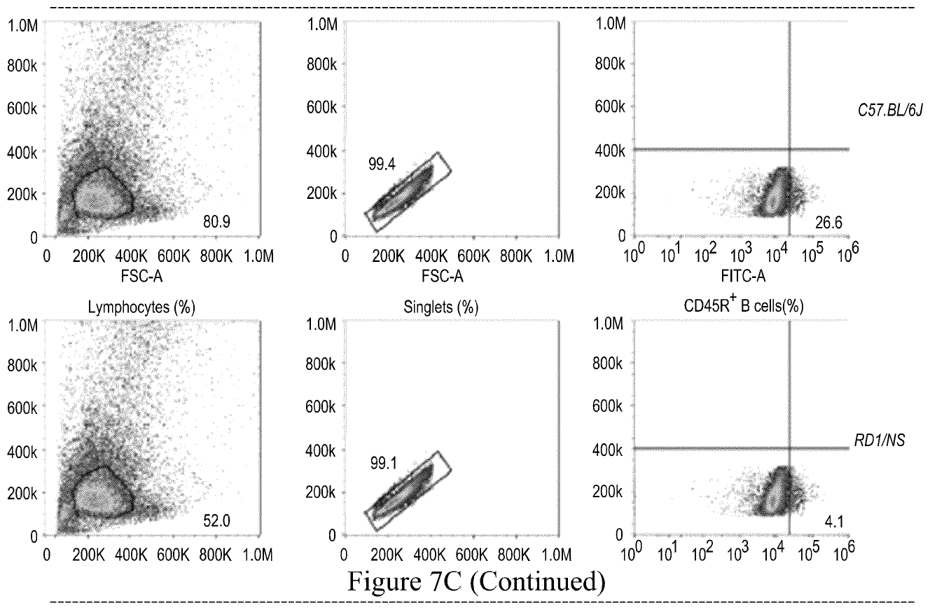

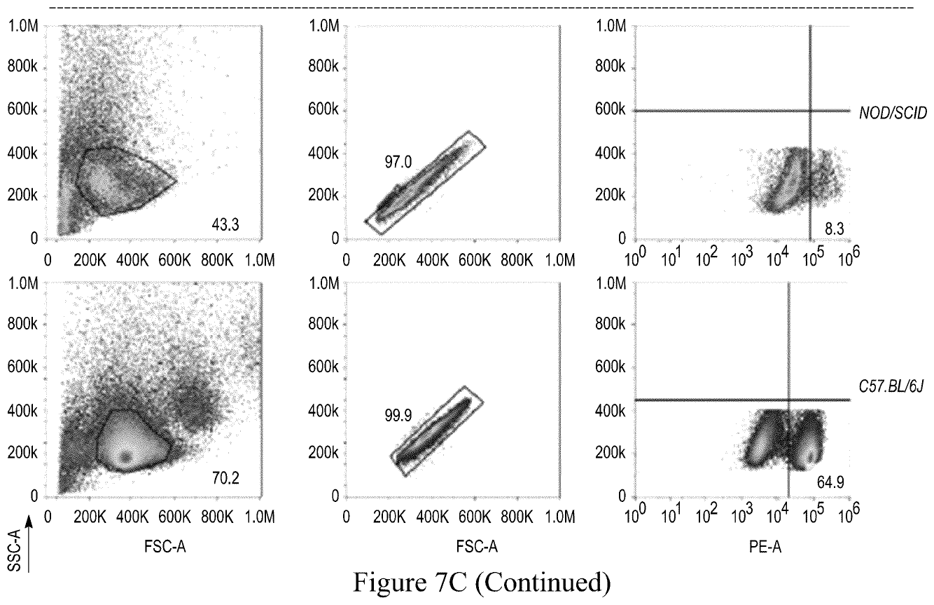

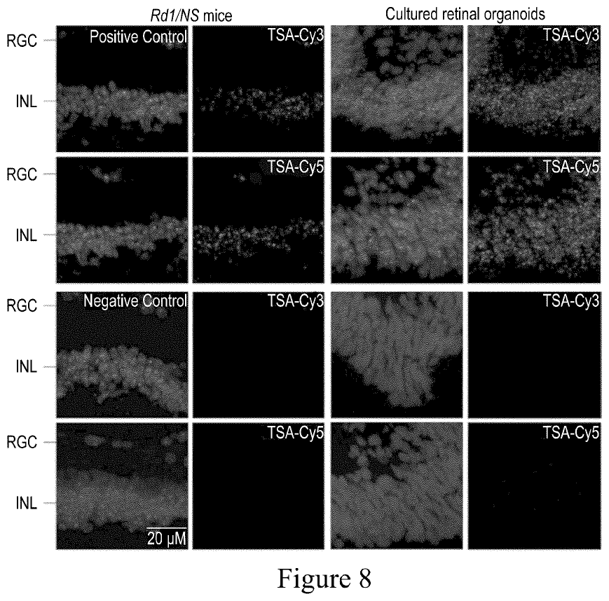

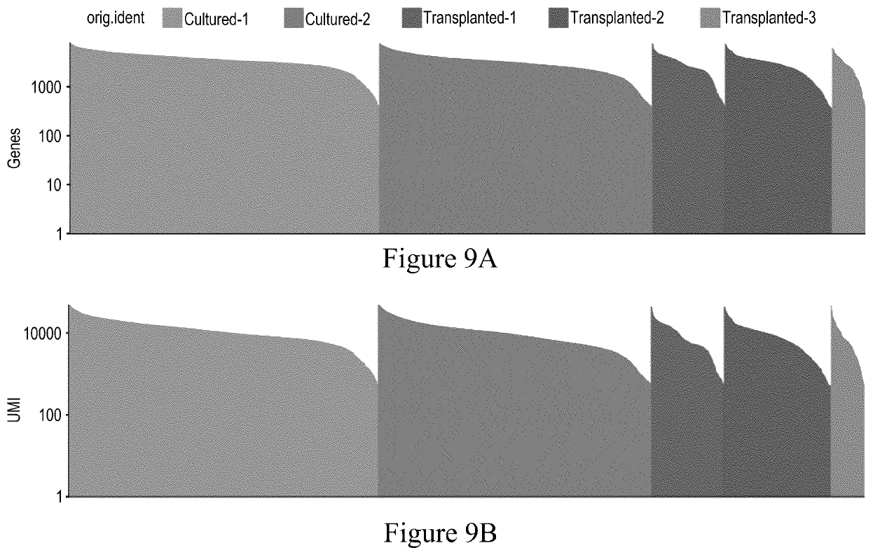

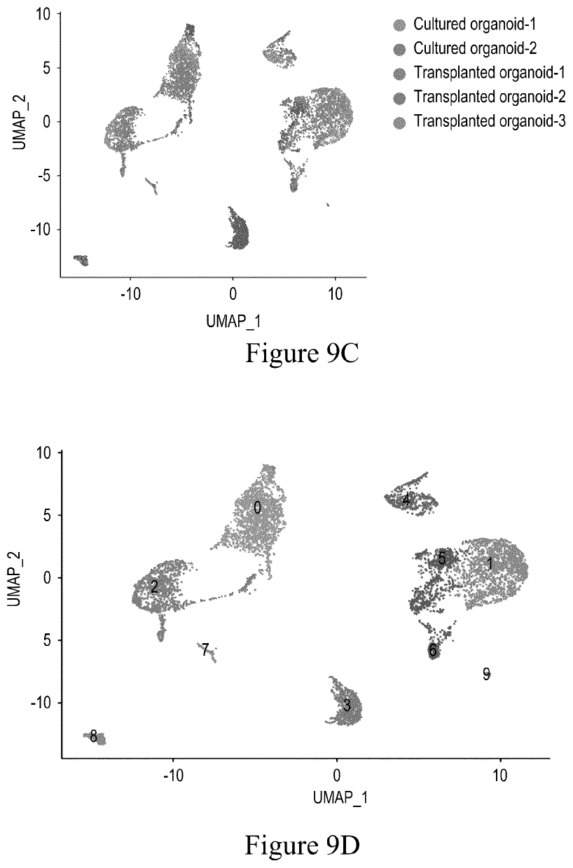

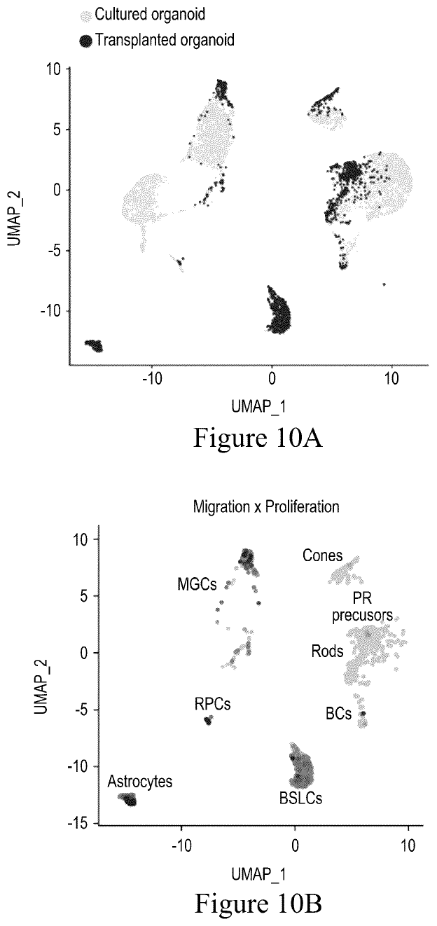

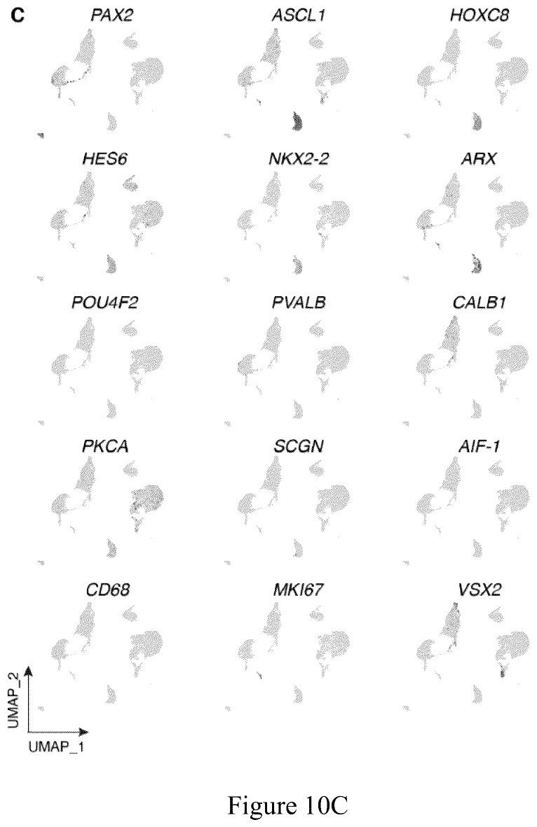

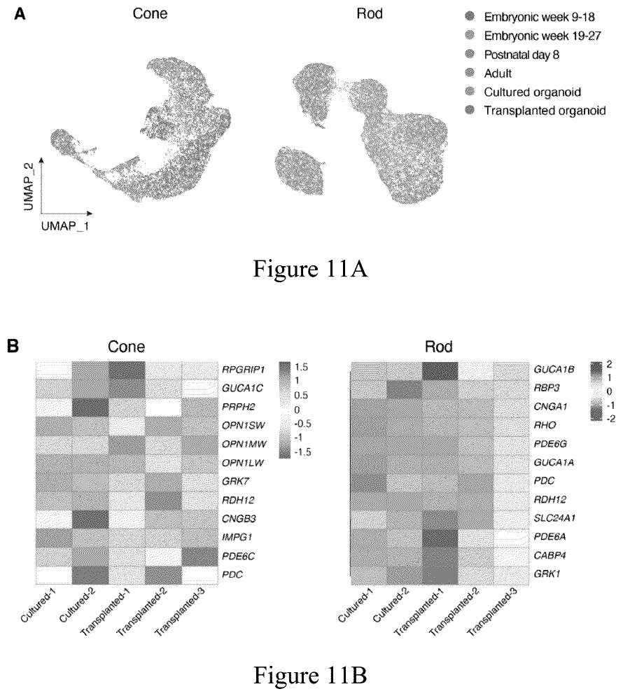

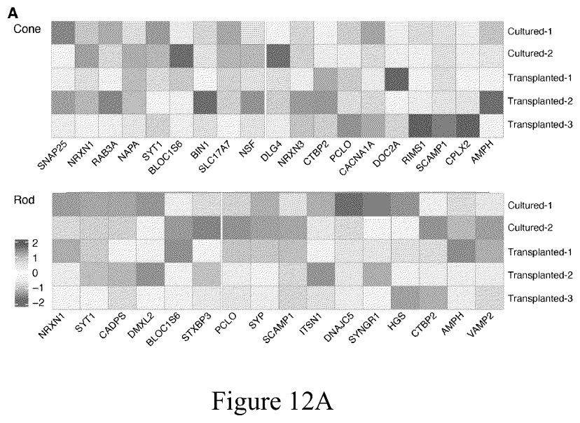

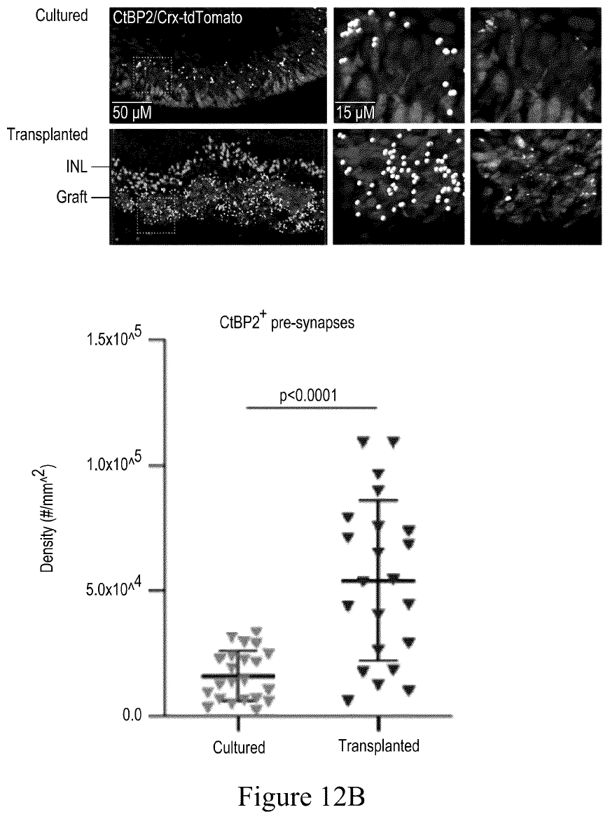

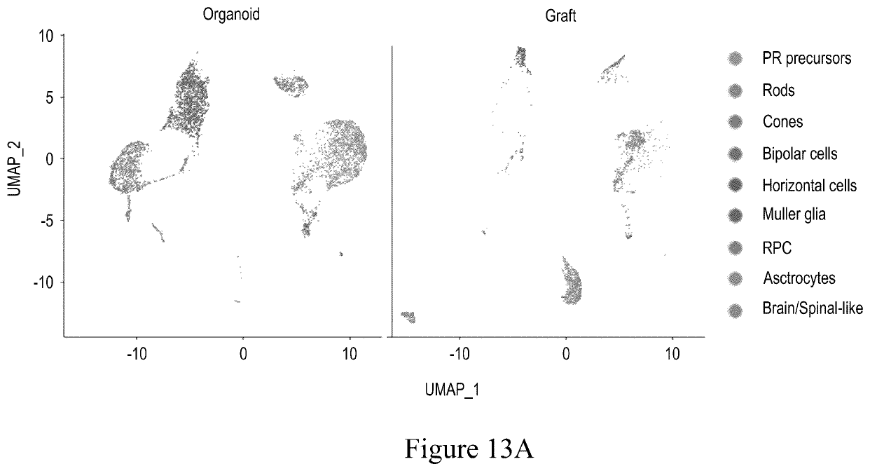

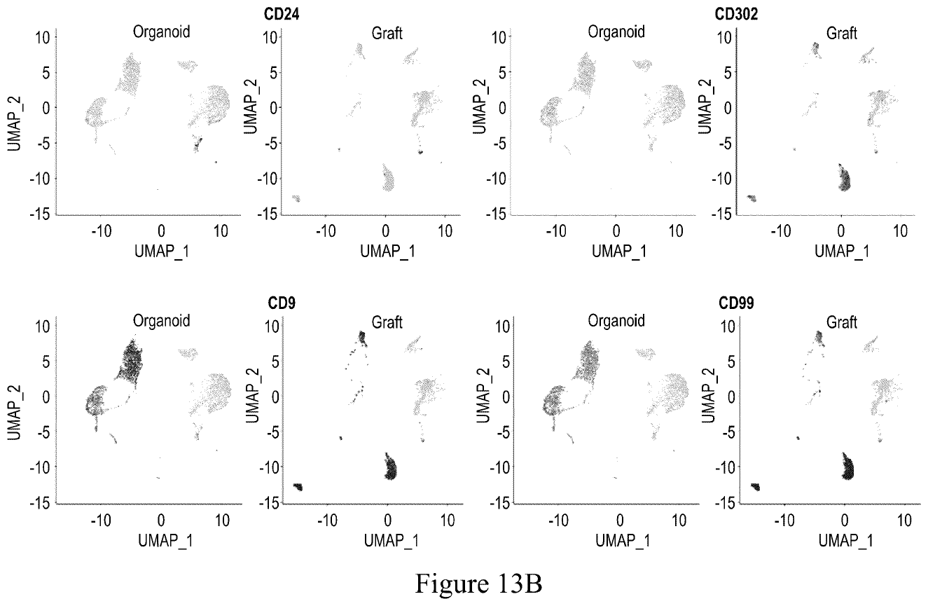

Next, we evaluated rod maturation in transplanted organoids and cultured organoids. As with cones, gene expression and pseudotime analysis suggested that rods from transplanted organoids resembled adult rods, whereas culture rods from cultured organoids resembled embryonic rods (FIG. 6A-B). Expression of RHO (FIG. 6C) and other rod-specific genes (Supplementary FIG. S5) was higher in rods from transplanted organoids than cultured organoids. The proportions of CRX:tdTomato⁺ cells that expressed Rho were significantly higher in transplanted organoids (61.5%) compared to cultured organoids (45.5%) (FIG. 6D). Similarly, the fraction of Rho⁺ cells with inner or outer segments was significantly higher in transplanted organoids (89.6%) than cultured organoids (29.8%) (FIG. 6E).

Finally, we investigated general features of photoreceptor maturity. Expression of certain synaptic proteins was upregulated in cones and rods in transplanted retinal organoids compared to cultured retinal organoids (Supplementary FIG. S6A). In CRX:tdTomato⁺ donor photoreceptors, the number of CtBP2⁺ puncta, which marks synaptic ribbons, was significantly higher in cells from transplanted organoids compared to cultured retinal organoids (Supplementary FIG. S6B). These data suggest that the recipient subretinal space, compared to the in vitro environment of cultured organoids, promotes maturation of rods and cones.

### 5. CD Markers for Selection/Purification of Therapeutic Cells

Digestion. Recognizing that donor cell viability is critical for transplantation outcome, a single-cell dissociation system was built optimal for the generation of a single cell suspension from retinal organoids. The digestion efficiency and cell viability were compared using Papain and Accumax Solution. Single cells from Papain digestion showed higher viability (90%) than Accumax solution (10%), while the Papain is much less efficient than Accumax (yield: Papain 0.6×10⁵ cells/h vs. Accumax 2×10⁵ cells/h). Agitation is used to approximately double the dissociation efficiency. An optimal agitation setting is 400 rpm for 3 hours at 37° C.

Storage conditions prior to transplantation. The storage conditions for dissociated donor cells prior to cell sorting and transplantation were optimized. Over 85% of cells in the suspension survived for at least 4 hours when maintained at 4° C., whereas cell survival after storage at 37° C. was less (approximately 70%).

Magnetic-assisted cell sorting (MACS). The donor retinal organoids are dissociated from relatively mature organoids (>120 days of differentiation, and up to 180 days or more). Less mature organoids (<55 days) containing only rare photoreceptors were used as the negative control. In mature retinal organoids, 12.5% of cells were CD24⁺CD99⁺ and 87.5% of cells were CD24⁻CD99⁻ cells. In less mature retinal organoids, 12.3% of cells dual-expressed CD24⁺ and CD99⁺. To validate the identity of sorted cells, we performed immunocytochemistry (ICC) using markers expressed in photoreceptor cells (anti-recoverin, REC) and glial cells including astrocytes (anti-glial fibrillary acid protein, GFAP). In the CD24⁻CD99⁻ cell suspensions derived from mature organoids, REC⁺ photoreceptors accounted for 48% of cells and GFAP⁺ cells accounted 37% of cells. In the CD24⁺CD99⁺ cell suspensions derived from mature organoids, we did not detect REC⁺ photoreceptors. In the CD24⁺CD99⁺ cell suspensions derived from less mature organoids, we did not detect REC⁺ photoreceptors.

### 6. Discussion

In these studies, we observed two major differences between cells from donor retinal organoids transplanted into mice and cells from chronologically equivalent retinal organoids maintained in culture. The transplanted cells were maintained in the degenerative recipient subretinal space for several months, therefore simulating conditions directly relevant for cell-based therapies for photoreceptor dystrophy. The most prominent and unexpected difference was the observation of migratory donor astrocytes and BSL cells in the transplanted cell population. Astrocytes and BSL cells underwent radial migration into, and long-distance tangential migration along, all retinal laminae (apart from the outer nuclear layer of photoreceptor cells that was absent in the degenerate recipients). The migratory astrocytes and BSL cells were generally non-proliferative, although graft-derived retinal progenitors showed proliferation without migration. In contrast to these migratory cells, transplanted photoreceptors, inner retinal neurons, and Müller glia were non-migratory and remained in the subretinal transplant site. The second major difference between transplanted and cultured organoids pertained to photoreceptor maturity. Based on gene expression and morphology, transplanted rods and cones were more mature than photoreceptors from cultured organoids. These data expand our understanding of photoreceptor and non-photoreceptor development in transplanted retinal organoids and highlight the importance of unbiased approaches to cell fate identification and spatial tracking following organoid transplantation.

The migratory astrocytes and BSL cells from transplanted organoids display molecular profiles distinct from cells in mature cultured organoids. The astrocytes express PAX2, which normally delineates the optic stalk in vivo. PAX2 is detected in retinal progenitors in early-stage retinal organoids but is undetectable at later stages. Moreover, cultured retinal organoids have not been reported to generate astrocytes in vitro. The BSL cells express ASCL1, HOXC8, NKX2-2, and ARX. ARX and NKX2-2-expressing cells are found in very early-stage retinal organoids, but not after 60 days in culture. HOXC8 expression is normally restricted to the posterior spinal cord and is absent from developing human retina and retinal organoids. Though PAX2⁺ astrocytes and ARX⁺ telencephalic interneurons undergo long-distance tangential migration in vivo, astrocyte or BSL identity was not sufficient to induce migration of graft-derived cells, as many astrocytes and BSL cells remained localized in the subretinal space. Our experiments lacked the temporal resolution to determine whether transplantation induced trans-differentiation of cells that initially adopted retinal identity or selectively promoted the proliferation of small numbers of residual BSL cells.

Prior publications have shown migratory transplanted cells, but their capacity to proliferate and migrate long distances were not known. Seiler and colleagues noted migratory human donor cells six months after transplantation of early-stage hESC-derived retinal organoids in the retinal degeneration nude rat. Using LMNB2 to identify human donor cells, Lamba and colleagues reported occasional migratory human induced pluripotent stem cell (iPSC)-derived PAX6⁺ and GFAP⁺ cells at 2 months. In another study, migratory cells were seen just seven days after subretinal delivery of human fetal CD29⁺ /SSEA1⁺ donor cells, suggesting that migration occurs soon after transplantation. Whether or not the early-migratory cells are the same as those observed months later is not yet known. In the wild-type cat, enhanced immunosuppression appeared to lead to greater cell migration, suggesting a role of immune cells. It is not known whether migratory donor cells negatively affect recipient retinal function and if depletion is required prior to transplantation.

The cues in the host environment that promote migratory cell fate and maturation of photoreceptors are not known. Multiple cell-extrinsic cues regulate cell specification in human retinal organoids. Dynamic regulation of thyroid hormone and retinoic acid signaling specifies cone subtypes in human retinal organoids. Though the roles of these cues in the subretinal environment following transplantation is not understood, they potentially regulate photoreceptor specification and maturation.

In conclusion, we found that human stem cell derived retinal organoid cells are affected by the murine host graft environment in two distinct ways. First, the host environment promotes a population of organoid-derived astrocytes that are capable of radial and tangential migration. Second, the host environment promotes the maturation of organoid-derived rod and cone photoreceptors that remain in the subretinal space.

### 7. Materials and Methods

a. Study Design

This study was designed to investigate the influence of recipient retinal microenvironment on human donor cell migration, fate specification, and maturation. Retinal organoids derived from CRX:tdTomato H9 human embryonic stem cells (hESC-H9) were adopted as donor cells and transplanted into Rd1/NS mice with retinal degeneration and immune deficiency. Four and a half months post-transplantation, migratory and non-migratory human donor cells were identified by single cell RNA sequencing (scRNA-seq) analysis and histological (i.e., RNAscope and immunohistochemistry counter staining). Lineage specification and maturation of donor human cells were characterized by scRNA-seq, pseudo-time series reconstitution, histological analysis, and electrophysiology recording. Age-matched human retinal organoids cultured in vitro served as controls.

b. Cell Culture and Retinal Organoids Differentiation

The use of human stem cells was approved by the Johns Hopkins ISCRO (ISCRO00000249). The CRX:tdTomato H9 human embryonic stem cell line (hESCs) was cultured following the gravity aggregation approach to differentiate retinal organoids, as previously described. On day 134, retinal organoids were used for transplantation.

The use of human stem cells was approved by the Johns Hopkins ISCRO (ISCRO00000249). The H9 CRX:tdTomato human embryonic stem cell line (hESCs) was a kind gift from Dr. David M. Gamm (University of Wisconsin Hospitals, USA). Stem cells were maintained in mTeSR1 (Stem Cell Technologies, Cambridge, MA, USA) on 1% (vol/vol) Matrigel-GFRTM (BD Biosciences, USA, No. 354230,) coated dishes and grown in a 37° C. HERAcell 150i incubator at 10% CO2 and 5% O2 incubator (Thermo Fisher Scientific, MA, USA). Cells were passaged upon confluence (every 3-6 days) using Accutase (Sigma-Aldrich, MO, USA, No. SCR005) for 7-10 minutes, and dissociated to single cells. Cells in Accutase were added 1:2 to mTeSR1 plus 5 μM Blebbistatin (Bleb; B0560, Sigma), pelleted at 80 g for 5 minutes, and suspended in mTeSR1 plus Bleb and plated at 5,000 cells per well in a six-well plate. After 48 hours, cells were fed with mTeSR1 (without Bleb) every 24 hours until the next passage. To minimize cell stress, no antibiotics were used in RPMI (Gibco, USA) and supplement media (10% fetal bovine serum (FBS), 2.5% penicillin). Cells were maintained at 37° C. and 5% CO2 and passaged every 3-4 days at ˜1×10\hat{ }5−2×10\hat{ }6 cells/mL in uncoated flasks. Cells were routinely tested for mycoplasma using MycoAlert (Lonza, Switzerland, No. LT07).

H9 CRX:tdtomato hESCs were dissociated in Accutase at 37° C. for 12 min and seeded in 50 μl of mTeSR1 at 3,000 cells/well into 96-well ultra-low adhesion round bottom Lipidure coated plates (AMSBIO, MA, USA, No. 51011610). Cells were placed in hypoxic conditions (10% CO2 and 5% O2) for 24 hours to enhance survival. Cells naturally aggregated by gravity over 24 hours. On day 1, cells were moved to normoxic conditions (5% CO2). On days 1-3, 50 μl of BE6.2 media, Supplementary Table 1) containing 3 μM Wnt inhibitor (IWR1e, EMD Millipore, MA, USA, No. 681669,) and 1% (v/v) Matrigel were added to each well.

TABLE 1
BE6.2 media for early retinal differentiation

| Reagent | Concentration | | Source | Catalog Number |
|---|---|---|---|---|
| DMEM | — | | Gibco | 11885084 |
| B27 Minus | 2% | | Gibco | 12587010 |
| vitamin A | | | | |
| Glutamax | 1% | | Gibco | 35050061 |
| NEAA | 1% | | Gibco | 11140050 |
| Sodium pyruvate | 1 | mM | Gibco | 11360070 |
| NaCl | 0.87 | mg/mL | Sigma-Aldrich | S9888 |
| E6 supplement | 2.5% | | | |
| Insulin | 970 | ug/mL | Roche | 11376497001 |
| Holo-transferrin | 535 | ug/mL | Sigma-Aldrich | T0665 |
| L-ascorbic acid | 3.20 | mg/mL | Sigma-Aldrich | A8960 |
| sodium selenite | 0.7 | ug/mL | Sigma-Aldrich | S5261 |

On days 4-9, 100 μl of media were removed from each well, and 100 uLs of media were added. On days 4-5, BE6.2 media containing 3 μM Wnt inhibitor and 1% Matrigel was added. On days 6-7, BE6.2 media containing 1% Matrigel was added. On days 8-9, BE6.2 media containing 1% Matrigel and 100 nM Smoothened agonist (SAG, EMD Millipore, No. 566660) was added. On day 10, aggregates were transferred to 15 mL tubes, rinsed 3× in DMEM (Gibco, No. 11885084), and resuspended in BE6.2 with 100 nM SAG in untreated 10 cm polystyrene petri dishes. From this point on, media was changed every other day. Aggregates were monitored and manually separated if stuck together or to the bottom of the plate. On day 11, retinal vesicles were manually dissected using sharpened tungsten needles. After dissection, cells were transferred into 15 mL tubes and washed 2× with 5 mLs of DMEM. On days 14-17, long-term retina (LTR, Supplementary Table 2) media with 100 nM SAG was added.

TABLE 2
Long-term retina media (LTR) for retinal organoids culturing

| Reagent | Concentration | Source | Catalog Number |
|---|---|---|---|
| DMEM | — | Gibco | 11555084 |
| F12 | 25% | Gibco | 11765062 |
| B27 | 2% | Gibco | 17504044 |
| NEAA | 1% | Gibco | 11140050 |
| Fetal bovine serum | 10% | Gibco | 16140071 |
| Sodium pyruvate | 1 mM | Gibco | 11360070 |
| Glutamax | 1% | Gibco | 35050061 |
| Taurine | 1 mM | Sigma-Aldrich | T-8691 |

On days 18-21, cells were maintained in LTR and washed 2× with 5 mLs of DMEM, before being transferred to new plates to wash off dead cells. To increase survival and differentiation, 1 μM all-trans retinoic acid (ATRA; R2625; Sigma) was added to LTR medium from days 22-138. 10 μM Gammasecretase inhibitor (DAPT, EMD Millipore, No. 565770) was added to LTR from days 28-42. Retinal organoids were grown at low density (10-20 per 10 cm dish) to reduce aggregation.
c. Recipient Mice

All animal experiments were carried out in accordance with the ARVO Statement for the Use of Animals in Ophthalmic and Vision Research. All procedures were approved by the Johns Hopkins University Animal Care and Use Committee (approval M016M17). We created a recipient mouse model with immune-deficiency and retinal degeneration (referred to as Rd1/NS) by crossbreeding Rd1 mice and Nod-Scid (NS) mice as previously reported. Mice were genotyped by Transnetyx Tag Center (Cordova, TN, USA), and characterized by immunohistochemistry (IHC) staining and flow cytometry analysis as previously reported.

The C3H/HeJ-Pde6brd1 (referred to as rd1), and NOD.CB17-Prkdcscid/J (referred to as NS) mice of either gender (aged 6 to 8 weeks) were obtained from Jackson Laboratory (Bar Harbor, ME, USA). All mice were housed in cages under a 12:12-hour light-dark cycle with water and food provided ad libitum.

We created a recipient mouse model with immune-deficiency and retinal degeneration (referred to as Rd1/NS) by crossbreeding Rd1 mice and Nod-Scid (NS) mice (aged 8 weeks). The breeding strategy was performed as previously reported. Genomic DNA of the third-generation offspring was extracted from ear biopsies and genotyped by Transnetyx Tag Center (Cordova, TN, USA). Primers were listed in Supplementary Table 3.

TABLE 3
Forward and reverse primer sequences
used for mice genotyping

| Gene | F primer (5′ to 3′) | R primer (5′ to 3′) |
|---|---|---|
| Rd1 | ACTCTGTGGCCTCAAAG | TGCAGGTCACAGAATCA |
| Wild | ATACATC | TCATAACA |
| type | (SEQ ID NO.: 1) | (SEQ ID NO.: 2) |
| Rd1 | GGGTCTCCTCAGATTGAT | GTCACTCTGTGGCCTCA |
| Mutant | TGACTAC | AAGAT |
| | (SEQ ID NO.: 3) | (SEQ ID NO.: 4) |
| NOD/ | TGTAACGGAAAAGAATTG | GTTGGCCCCTGCTAACTT |
| SCID | GTATCCACA | TCT |
| | (SEQ ID NO.: 5) | (SEQ ID NO.: 6) |

Eyes of adult Rd1/NS mice (n=3) were collected to characterize photoreceptor degeneration using immunohistochemistry (IHC) staining, as previously reported. Flow cytometry was performed using spleen biopsies of adult Rd1/NS mice (n=3) to confirm the deficiency of T cells and B cells, as previously described. Phenotyping data of Rd1/NS mice are shown in Supplementary FIG. S1.
d. Transplantation

Donor retinal organoid cells (harvested as micro-dissected multilayered retinal fragments) were obtained from Crx:tdTomato⁺ hESC-derived retinal organoids (aged 134 days, n=4). The cultured human retinal organoids were imaged using a fluorescent microscope (Carl Zeiss, Jena, Germany). The images were used as a reference to isolate the Crx-tdTomato⁺ cluster from the donor retinal organoids. The isolated Crx-tdTomato⁺ retinal organoids clusters were then cut into 1×1 mm\hat{ }2 or 1×2 mm\hat{ }2 microdissected sheets using a 27-gauge horizontal curved scissors (VitreQ, Kingston, NH, USA) under a dissection microscope. Donor cells were transplanted within two hours of isolation.

The isolated retinal fragments were transplanted into the subretinal space of Rd1/NS mice (aged 6 to 8 weeks, n=16 eyes), as previously reported. Briefly, recipient mice were anesthetized by intraperitoneal injection of ketamine (100 mg/kg body weight) and xylazine hydrochloride (20 mg/kg body weight). Mouse pupils were dilated with 1% (wt/vol) tropicamide (Bausch & Lomb, Rochester, NY, USA). Mouse corneas were covered with Sodium Hyaluronate (Healon GV, Abbott Medical Optics Inc. CA, USA) and cover glasses (Deckglaser, USA) to facilitate transpupillary visualization. The donor retinal organoid sheets were loaded into the bevel of a 26G microneedle with the photoreceptor side facing down, gently aspirated into the attached microsyringe (Hamilton, Reno, NV, USA), then tangentially injected into the subretinal space through the sclera of the recipient mice. Successful injection was verified by direct visualization through the dilated pupil of the recipient under the surgical microscope (Leica, Wetzlar, Germany).

e. Single Cell RNA Sequencing

Four and a half months post-transplantation, single cell RNA sequencing (scRNA seq) was performed on dissociated single cells from transplanted (n=3 eyes) and cultured retinal organoids (n=2) (age-matched) using the Chromium platform (10× Genomics). ScRNA-Seq was performed on dissociated cells from transplanted and cultured retinal organoids using the Chromium platform (10× Genomics). Briefly, retinal organoid cells were dissociated into a single cell suspension using the Papain Dissociation System (Worthington) for 60 minutes 31 at 37° C., with gentle mixing every 5 minutes, before stopping the reaction using ovomucoid protease inhibitors. Cells were centrifuged and resuspended in ice-cold PBS containing 0.04% bovine serum albumin (BSA) and 0.5 U/μl RNase inhibitor and were filtered through a 40-μm Flowmi cell strainer (Bel-Art SP). Cell counts and viability was assessed by Trypan blue staining before loading 6000 cells on a Chromium Single Cell system using Next GEM 3′ reagent v3.1 kits. Libraries were pooled and sequenced on Illumina NextSeq 500 with ˜50,000 reads per cell. The Cell Ranger 4 (10× Genomics) pipeline was used to process the raw sequencing reads for demultiplexing, alignment to the GRCh38 human reference genome and generating the cell-bygene count matrix for downstream analysis. The generated cell-by-gene count matrices were analyzed using the Seurat ver3 R package. We filtered out cells that had UMIs less than 300 or greater than 50000 and with a mitochondrial fraction of greater than 20%. Doublets were identified and removed using the DoubletFinder R package. Log-normalization, scaling, UMAP dimensional reduction and clustering were performed using the standard Seurat pipeline. Major retinal cell types were identified using previously identified cell type markers. Enriched genes from the brain/spinal-like cell cluster were compared to the ASCOT gene expression summaries of public RNA-Seq data to determine its classification. Differential gene tests were performed by Seurat's FindMarkers function using the Wilcoxon rank sum test with default parameters (52). Hierarchical clustering was used to group the differentially expressed genes. The UCell R package was used to calculate the migration potential score or the proliferation score. The gene sets were constructed by identifying enriched genes within the gene ontology terms cell migration and cell motility for the migration potential score and cell division for the proliferation score respectively. The Seurat integration functions (SelectIntegrationFeatures, FindIntegrationAnchors and IntegrateData) were used to integrate the organoid data onto the human retinal developmental dataset. Monocle 3 was used to perform pseudotime analysis and identify trajectory routes within the data.

f. Histological Analysis

Four and a half months post-transplantation, the recipient mice eyes and cultured retinal organoids were fixed with 4% paraformaldehyde (PFA) (Electron Microscopy Sciences, Hatfield, PA, USA) in PBS and dehydrated in a sucrose gradient (10%, 20%, 30%), then blocked in optimal cutting temperature compound (OCT) (Sakura Finetek, Torrance, CA, USA). Seven to ten micrometer sections of recipient eyes and cultured organoids were used for RNAscope and IHC counter staining.

The recipient mice were sacrificed with over-dose anesthesia and pre-fixed by heart-perfusion with 4% paraformaldehyde (PFA) (Electron Microscopy Sciences, Hatfield, PA, USA) in PBS. Eyes were gently removed, post-fixed in 4% PFA/PBS for one hour at room temperature (RT), and dehydrated in a sucrose gradient (10%, 20%, 30%), then blocked in optimal cutting temperature compound (Sakura Finetek, Torrance, CA, USA). Cultured retinal organoids were fixed in 4% PFA at RT for 15 minutes (min), dehydrated in gradient sucrose (10%, 20%, 30%), and blocked in the OCT compound. OCT-blocked recipient mouse eyes and cultured retinal organoids were cut into 7-10 μm thick cryosections using a microtome (CM 1850; Leica) for histological staining. RNAscope and IHC counter-staining was performed according to the manufacturer's protocol (Advanced Cell Diagnostics (ACD), see Protocol #MK 51-150, Appendix D.). Briefly, cryosections of recipient mice eyes and cultured retinal organoids were rinsed with PBS, baked in a HybEZ™ oven (ACD, USA) for 30 min at 60° C., and post-fixed in pre-chilled 4% PFA in PBS for 15 min at 4° C. Slides were dehydrated in gradient ethanol (50%, 70%, 100%), treated with hydrogen peroxide (10 min at RT), then subjected to target retrieval using the Co-detection Target Retrieval solution (ACD, Cat. No. 323180) at 98-102° C. for 5 min. After rinsing in distilled water (2 min×2) and PBS-T (5 min×1), the slides were incubated with diluted primary antibody at 4° C. overnight. On day 2, slides were post-fixed with 4% PFA for 30 min at RT, treated with protease III at 40° C. for 30 min, and subjected to RNAscope staining using the RNAscope Multiplex 32 Fluorescent V2 assay according to the manufacturer's protocol (ACD, RNAscope USM-323100, see “fixed-frozen tissue sample protocol”). Briefly, RNA probe hybridization was performed with the HybEZ™ oven for two hours at 40° C. Slides were then assigned for three series of amplification, fluorochromes combination, and HRP blocking. After the RNAscope procedure, slides were incubated with secondary antibody at RT for one hour, counter-stained with DAPI, and mounted with Prolong Diamond (Life Technology, Carlsbad, CA, USA). The RNA probes, fluorophores, primary antibodies, and secondary antibodies used were listed in Supplementary Table 4.

TABLE 4
Reagents used for RNAscope and immunohistochemistry
counter-staining

| Reagents | Dilution | Source | Catalog Number |
|---|---|---|---|
| RNA probes | | | |
| PAX2-Hs | No dilution | Advanced Cell | 442541 |
| | | Diagnostics | |
| HES6-Hs | 1:50 | Advanced Cell | 521301-C2 |
| | | Diagnostics | |
| ASCL1-Hs | 1:50 | Advanced Cell | 459721-C2 |
| | | Diagnostics | |
| NKX2-2-Hs | No dilution | Advanced Cell | 821401 |
| | | Diagnostics | |
| HOXC8-Hs | No dilution | Advanced Cell | 506531 |
| | | Diagnostics | |
| ARX-Hs | 1:50 | Advanced Cell | 486711-C2 |
| | | Diagnostics | |
| VSX2-Hs | 1:50 | Advanced Cell | 493031-C2 |
| | | Diagnostics | |
| 3-plex positive | No dilution | Advanced Cell | 320881 |
| control-Mm | | Diagnostics | |
| 3-plex positive | No dilution | Advanced Cell | 320861 |
| control-Hs | | Diagnostics | |
| 3-plex negative | No dilution | Advanced Cell | 320871 |
| control | | Diagnostics | |
| TSA-fluorophores | | | |
| TSA plus-Cy3 | 1:1500 | AKOYA | NEL744001KT |
| | | Biosciences | |
| TSA plus-Cy5 | 1:1500 | AKOYA | NEL745001KT |
| | | Biosciences | |
| Primary antibodies | | | |
| Sheep anti-Ki67 | 1:40 | R&D Systems | AF7617 |
| Rabbit anti-Ku80 | 1:50 | Thermo Fisher | MA5-32212 |
| | | Scientific | |
| Secondary antibodies | | | |
| Donkey anti- | 1:200 | Thermo Fisher | A-21448 |
| Sheep 647 | | Scientific | |
| Donkey anti- | 1:200 | Thermo Fisher | A21206 |
| Rabbit 488 | | Scientific | |

Negative and positive multiplex control probes staining were run in parallel with the target probes following the same protocol (data shown in Supplementary FIG. S2). IHC staining was performed as previously described. Briefly, cryosections of transplanted Rd1/NS mice and cultured retinal organoids were rinsed with PBS (5 min×1), permeabilized, and blocked with a mixture of 0.1% Triton-X100 and 5% goat serum in PBS for one hour at RT. The slides were rinsed in PBS (5 min×3), incubated with primary antibodies at 4° C. overnight, incubated with secondary antibodies at RT for one hour, then counter stained with DAPI and mounted using ProLong Diamond mounting media. The primary antibodies and secondary antibodies used were listed in Supplementary Table 5.

TABLE 5
Antibodies used for immunohistochemistry staining

| Reagents | Dilution | Source | Catalog Number |
|---|---|---|---|
| Primary antibodies | | | |
| Rabbit anti-RBPMS | 1:500 | MilliporeSigma | ABN1362 |
| Mouse anti-NeuN | 1:500 | MilliporeSigma | MAB377 |
| Mouse anti-Calbindin | 1:500 | Sigma-Aldrich | C9848 |
| Goat anti-PKCα | 1:200 | R&D Systems | AF5340 |
| Goat anti-SCGN | 1:200 | Thermo Fisher | PA5-47664 |
| | | Scientific | |
| Rabbit anti-IBA1 | 1:250 | Abcam | Ab178680 |
| Rabbit anti-CD68 | 1:100 | Abcam | Ab125212 |
| Rabbit anti-Ki67 | 1:500 | Thermo Fisher | MA5-14520 |
| | | Scientific | |
| Rabbit anti-L/M-opsin | 1:500 | Kerafast | EDK101 |
| Rabbit anti-S-opsin | 1:500 | MilliporeSigma | Ab5407 |
| Rabbit anti-Rhodopsin | 1:500 | Abcam | Ab3424 |
| Mouse anti-human | 1:1000 | MilliporeSigma | Mab1281 |
| nuclear antibody (HNA) | | | |
| Secondary antibodies | | | |
| Goat anti-Rabbit 488 | 1:500 | Abcam | Ab150077 |
| Goat anti-Mouse 488 | 1:500 | Thermo Fisher | A-11001 |
| | | Scientific | |
| Goat anti-Mouse 647 | 1:500 | Thermo Fisher | A32728 |
| | | Scientific | |
| Donkey anti-Goat 488 | 1:500 | Abcam | Ab150129 |

For migratory distance quantification of transplanted retinal organoid cells, retinal sections from recipient mice were stained with human nuclear specific antibodies HNA (Sigma-Aldrich, MO, USA) or Ku80 (Thermo Fisher Scientific, MA, USA). Tile scan images were collected using Confocal LSM 880 (Zeiss, Oberkochen, Germany) for distance quantification. The migratory distance of transplanted retinal organoids was defined as the shortest distance between the migratory cells and the nearest graft edge (i.e., the graft-left migratory cells to the left endpoint of the graft; the graft-right migratory cells to the right endpoint of the graft). We used a mathematical method to facilitate distance quantification. Specifically, the graft edge was defined as a “starting point” and the migratory cells in different retinal laminae (RGC, IPL, INL, RPE/C) were manually targeted, both processed with the “Cell Counter” plugin in ImageJ. The cell coordinates were automatically collected to quantify the X and Y axial distances of individual cells by the Cell Counter. The axial distance of the graft edge (starting point) was referred to as “X start” and “Ystart”. The axial distance of the migratory cells was referred to as “X migratory” and “Ymigratory”. The migratory distance was computed in R platform following the formula:

$$Migrating distance = \sqrt{{( {X}_{start} - {X}_{migrating} )}^{2} - {( {Y}_{start} - {Y}_{migrating} )}^{2}}$$

The unit of the migratory distance was converted from pixel to micron according to the image scale. For cell quantification, the number of positively stained cells was manually counted using the “Cell Counter” plugin in ImageJ. The representative pre-synapse graphs of the transplanted and cultured retinal organoids were drawn by Imaris software (Version 9.5.0, Bitplane AG, Zurich, Switzerland).
g. Electrophysiology

The electrophysiological recording was performed on the transplanted photoreceptors eight months post-transplantation to measure their physiological properties. We were able to test only one recipient mouse (the second recipient mouse died before the assay during the long-term observation). The recipient's eyes were gently pulled out from the recipient mouse and put in Ames' medium (Sigma No. A1420). Retinas with transplanted retinal organoids were dissected under infrared light and sectioned into 200 μm slices, then transferred to a recording chamber. The Crx:tdTomato⁺ photoreceptors of the transplanted retinal organoids were targeted under an epifluorescence microscope for consequent whole-cell patch-clamp recording. Fluorescent signal was imaged by a Nikon CCD camera with data acquisition synchronized with a 20-ms flash of epi-fluorescence excitation light. The total exposure time to excitation light before recording was <500 ms. During recording, retina was perfused with Ames' medium bubbled with 95% O2/5% CO2. Patch electrodes (5-7 MΩ) were pulled from borosilicate capillaries (GC150-10, Harvard Apparatus) and filled with an internal solution containing typically (in mM): 120 K-gluconate, 5 NaCl, 4 KCl, 10 HEPES, 2 EGTA, 4 ATP-Mg, 0.3 GTP-Na2, and 7 Phosphocreatine-Tris, with pH adjusted to 7.3 with KOH. Wholecell patch-clamp recording was made at 30-32° C. with an Axon Instruments Multiclamp 700B amplifier. Series resistance of patch electrodes was 10-30 MΩ. Liquid-junction potential (measured to be −13 mV) has been corrected. In voltage-clamp mode, recorded cells were held at −40 mV, followed by voltage steps of 100-ms (−70 mV to −10 mV). All procedures were carried out in the darkroom to avoid photoreceptor bleaching

h. Statistical Analysis

Quantitative histology data were analyzed using two-way ANOVA. Sidak's test or Tukey's test was adopted for multiple comparisons (two-tailed). Independent T-test or Mann-Whitney U test 11 was used for two variants comparison. Statistical analysis was carried out using SPSS software (version 25, IL, USA). p<0.05 was taken to be significant. Graphs were drawn with GraphPad Prism software (version 8, CA, USA).

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. As one of ordinary skill in the art will readily appreciate from the present disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the present disclosures of which are incorporated herein by reference in their entireties for all purposes.

What is claimed is:

1. An in vitro method to produce sorted population of retinal cells, comprising:
a) generating a three-dimensional retinal organoid;
b) dissociating the three-dimensional retinal organoid; and
c) positively sorting retinal cells based on one or more marker of photoreceptor cell identity and/or negatively sorting retinal cells based on one or more marker of non-photoreceptor cell identity to produce the sorted population of retinal cells.

2. The method of claim 1, wherein:
a) the marker of photoreceptor cell identity is CD73;
b) the marker of non-photoreceptor cell identity is one or more of CD24, CD302, CD9, and CD99; or
c) the marker of non-photoreceptor cell identity is one or more of ITM2B; CD63; ENO1; CALR; CANX; CLU; SLC3A2; BSG; GPM6B; ITGB1; PTTG1IP; TIMP1; PMEPA1; SSR2; DKK3; LRP1; ATRAID; HLA-A; HLA-C; EMP3; TMED9; GOLIM4; LTBP3; GALNT1; CD151; PLD3; CALU; LSAMP; CD59; SLC2A1; LAMP2; HLA-B; COL11A1; DPP7; DCBLD2; CD164; SLC1A3; F3; CTSD; FLNA; SLC39A10; FN1; TMEM106C; TMEM179B; ATP1B3; HLA-E; TMEM132A; FLT1; FGFR1; CAPNS1; FAT1; ANGPTL1; LRP10; CRELD2; SPPL2A; TSPAN4; PRSS35; ECE1; SYPL1; SORCS2; COL2A1; DNER; COL6A1; CD44; GPC1; PCDH9; CRIM1; CHL1; TTYH3; IKBIP; NECTIN2; FBLN2; CCDC80; DAG1; PBXIP1; PRSS23; ACAA1; NTRK2; FSTL1; BCHE; TNFRSF1A; LGALS3BP; ITGB8; CP; ADGRG1; VCAN; OLFM1; NRP1; SCARB1; TSPAN6; PCDH17; PLTP; NECTIN3; PTPRD; CADM4; UNC5B; CSPG5; AXL; PLXNB2; PLPP3; NOTCH2; SLITRK2; AEBP1; ANGPTL4; COLEC12; VAMP5; NLGN4X; FGFRL1; EFNB2; COL5A1; LAMB2; LAMC1; IGFBP3; FNDC5; FCGRT; ADRA2C; SERPING1; EPHB2; CDH11; COL1A2; CNTFR; AGRN; ROBO1; LOX; MRC2; COL6A2; SLC6A11; DSC2; IGSF8; EMP1; ABI3BP; TTYH2; NOTCH1; ANO6; A2M; SORCS1; EFNA1; PTPRG; TF; EMP2; CEMIP2; SERPINE2; CDON; EGFR; PCDH7; MAN2A1; IL1R1; COL1A1; SEMA5A; ANTXR1; S1PR3; ITPRIP; MXRA8; PRELP; AQP1; CSF1; BCAN; ADGRA2; CA12; FAT3; HEPACAM; FGFR3; TRIL; HSD17B2; and HP.

3. The method of claim 1 wherein the marker of non-photoreceptor cell identity is one or more of DKK3; LRP1; CLU; PMEPA1; ITGB1; and PTTG1IP.

4. The method of claim 1 wherein the one or more marker of non-photoreceptor cell identity is one or more astrocyte marker selected from ITM2B; CD63; ENO1; CALR; CANX; CLU; SLC3A2; BSG; GPM6B; ITGB1; PTTG1IP; TIMP1; PMEPA1; SSR2; DKK3; LRP1; ATRAID; HLA-A; HLA-C; EMP3; TMED9; GOLIM4; LTBP3; GALNT1; CD151; PLD3; CALU; LSAMP; CD59; SLC2A1; LAMP2; HLA-B; COL11A1; DPP7; DCBLD2; CD164; SLC1A3; F3; CTSD; FLNA; SLC39A10; FN1; TMEM106C; TMEM179B; ATP1B3; HLA-E; TMEM132A; FLT1; FGFR1; CAPNS1; FAT1; ANGPTL1; LRP10; CRELD2; SPPL2A; TSPAN4; PRSS35; ECE1; SYPL1; SORCS2; COL2A1; DNER; COL6A1; CD44; GPC1; PCDH9; CRIM1; CHL1; TTYH3; IKBIP; NECTIN2; FBLN2; CCDC80; DAG1; PBXIP1; PRSS23; ACAA1; NTRK2; FSTL1; BCHE; TNFRSF1A; LGALS3BP; ITGB8; CP; ADGRG1; VCAN; OLFM1; NRP1; SCARB1; TSPAN6; PCDH17; PLTP; NECTIN3; PTPRD; CADM4; UNC5B; CSPG5; AXL; PLXNB2; PLPP3; NOTCH2; SLITRK2; AEBP1; ANGPTL4; COLEC12; VAMP5; NLGN4X; FGFRL1; EFNB2; COL5A1; LAMB2; LAMC1; IGFBP3; FNDC5; FCGRT; ADRA2C; SERPING1; EPHB2; CDH11; COL1A2; CNTFR; AGRN; ROBO1; LOX; MRC2; COL6A2; SLC6A11; DSC2; IGSF8; EMP1; ABI3BP; TTYH2; NOTCH1; ANO6; A2M; SORCS1; EFNA1; PTPRG; TF; EMP2; CEMIP2; SERPINE2; CDON; EGFR; PCDH7; MAN2A1; IL1R1; COL1A1; SEMA5A; ANTXR1; S1PR3; ITPRIP; MXRA8; PRELP; AQP1; CSF1; BCAN; ADGRA2; CA12; FAT3; HEPACAM; FGFR3; TRIL; HSD17B2; and HP.

5. The method of claim 4 wherein the one or more marker of non-photoreceptor cell identity is one or more astrocyte marker selected from ADGRL4; SERPINE2; BCHE; ABI3BP; NRP1; FSTL1; FAT1; NTRK2; FBLN2; PRSS35; SLC1A3; FCGRT; LAMC1; TF; SORCS2; DKK3; LRP1; PTPRD; ANGPTL1; LTBP3; CLU; CNTNAP2; CD151; PCDH9; CRIM1; CSPG5; and PMEPA1.

6. The method of claim 1 wherein the one or more marker of non-photoreceptor cell identity is one or more brain and spinal cord-like (BSL) cell marker selected from CLU; ITM2B; PTPRZ1; GPM6B; ATP1B2; CD63; BCAN; SLC1A3; SERPINE2; LRP1; PTPRA; ADGRG1; ENO1; CANX; SLC3A2; DNER; PTTG1IP; CALR; PCDH9; CCDC80; LSAMP; HEPACAM; F3; PLPP3; APLP2; FBLN2; TIMP1; SLC6A11; CSPG5; JAM2; FGFR3; DKK3; GOLIM4; NCAM1; CHL1; NRCAM; HLA-A; TMEM132A; PMEPA1; ITGAV; SSR2; ACAA1; BCHE; CD59; FAT3; PCDH17; ST3GAL5; PBXIP1; LAMP1; ITGB1; HP; ITGB8; SGCB; LAMP2; CLDND1; TMEM106B; PTCH1; PLTP; RNF13; HLA-C; PTPRD; TMEM30A; TRIL; RAB5C; TTYH3; DAG1; CADM4; UBA1; SLC6A9; LRRC8A; ATP1B3; SPPL2A; NTRK2; RNF130; LIFR; EMP3; PCDH7; NTRK3; COL6A1; IL17D; LRP10; ADAM19; SGCE; FAT1; SLC44A1; LTBP3; SLC39A10; ABCA1; SYPL1; SLITRK2; GNPTG; CD302; MRC2; LRP4; CALU; CD151; SORL1; TSPAN6; LRRN1; TENM2; CAPNS1; NLGN1; SLC15A2; NLGN4X; EGFR; ADORA1; SLC9A7; SIRPA; EFCAB14; ANGPTL1; FGFR1; VAMP5; CLDN12; LAMB2; GPR155; FGFR2; SLC44A2; LRP1B; PTGFRN; FNDC5; NOTCH1; DPY19L4; S1PR1; CD44; TNFRSF1A; FAM234A; CDH4; HLA-E; COL11A1; NCAM2; AQP1; CPQ; EMP1; FCGRT; GPC5; ROBO1; VCAN; LGALS3BP; LDLR; LRRC4B; NOTCH2; ALCAM; RYR3; SLC9A9; TMEM94; VCAM1; IGSF1; PCDHA10; CDH10; CACHD1; P2RX7; AEBP1; PLXNB1; AXL; ALPL; ST3GAL4; SERPINI1; ITGB5; CD58; FGFRL1; PLPP1; TTYH2; IL17RB; FAM171A1; IL17RD; ANO6; ADAM22; PTPRG; ANTXR1; ZDHHC23; AGRN; COL14A1; POSTN; CNTFR; SEMA5A; FLNA; EMP2; TFPI; ITGA7; MXRA8; TENM4; FSTL1; CD82; NRP2; GPC4; ARSF; LAMC1; KIT; SEMA4A; LTBP1; and CSF1.

7. The method of claim 6 wherein the one or more marker of non-photoreceptor cell identity is one or more BSL marker selected from HEPACAM; FGFR3; SERPINE2; BCAN; CCDC80; PLPP3; CHL1; ADGRG1; SLC6A11; LSAMP; FBLN2; F3; SLC1A3; DKK3; LRP1; DNER; CLU; PCDH9; and CSPG5.

8. The method of claim 1, wherein the three-dimensional retinal organoid is enzymatically dissociated.

9. The method of claim 8, wherein the enzyme is papain and/or trypsin.

10. The method of claim 8, wherein the retinal cells are contacted with a composition to ensure that the cells remain in a dissociated cell suspension.

11. The method of claim 10, wherein the composition comprises an enzyme.

12. The method of claim 1, wherein the three-dimensional retinal organoid reaches between about DD 45 and DD 300 prior to being dissociated.

13. The method of claim 12, wherein the three-dimensional retinal organoid reaches about DD 90 to about DD 140 prior to being dissociated.

14. The method of claim 1, wherein the retinal cell population consists of at least about 70% single cells.

15. The method of claim 1, wherein the retinal cell population comprises about 55% to about 85% rod photoreceptor cells.

16. The method of claim 1, wherein the stem cells are selected from pluripotent or multipotent stem cells; human, nonhuman primate or rodent nonembryonic stem cells; human, nonhuman primate or rodent embryonic stem cells; human, nonhuman primate or rodent induced pluripotent stem cells; and human, nonhuman primate or rodent recombinant pluripotent cells.

17. A sorted population of in vitro differentiated retinal cells, wherein said in vitro differentiated retinal cells are obtained by a method comprising:
a) generating a three-dimensional retinal organoid;
b) dissociating the three-dimensional retinal organoid; and
c) positively sorting retinal cells based on one or more marker of photoreceptor cell identity and/or negatively sorting retinal cells based on one or more marker of non-photoreceptor cell identity to produce the sorted population of retinal cells.

18. A method of preventing and/or treating an inherited or acquired retinal degenerative disease in a subject, comprising administering to the subject an effective amount of in vitro differentiated retinal cells obtained by a method comprising:
a) generating a three-dimensional retinal organoid;
b) dissociating the three-dimensional retinal organoid; and
c) positively sorting retinal cells based on one or more marker of photoreceptor cell identity and/or negatively sorting retinal cells based on one or more marker of non-photoreceptor cell identity to produce the sorted population of retinal cells.

19. The method of claim 18, wherein the inherited retinal degenerative disease is selected from retinitis pigmentosa, choroideremia, Stargardt disease, cone-rod dystrophy, and Leber Congenital Amaurosis.

20. The method of claim 18, wherein the acquired retinal degenerative disease is age-related macular degeneration.


SEQUENCE LISTING (6 sequences)

SEQ ID NO: 1 - DNA, 24 bp, synthetic construct
  source 1..24: mol_type = other DNA
  misc_feature 1..24: note = Description of sequence: Rd1 Wildtype F primer
actctgtggcctcaaagatacatc

SEQ ID NO: 2 - DNA, 25 bp, synthetic construct
  source 1..25: mol_type = other DNA
  misc_feature 1..25: note = Description of sequence: Rd1 Wild type R primer
tgcaggtcacagaatcatcataaca

SEQ ID NO: 3 - DNA, 25 bp, synthetic construct
  source 1..25: mol_type = other DNA
  misc_feature 1..25: note = Description of sequence: Rd1 Mutant F primer
gggtctcctcagattgattgactac

SEQ ID NO: 4 - DNA, 22 bp, synthetic construct
  source 1..22: mol_type = other DNA
  misc_feature 1..22: note = Description of sequence: Rd1 Mutant R primer
gtcactctgtggcctcaaagat

SEQ ID NO: 5 - DNA, 27 bp, synthetic construct
  source 1..27: mol_type = other DNA
  misc_feature 1..27: note = Description of sequence: NOD/SCID F primer
tgtaacggaaaagaattggtatccaca

SEQ ID NO: 6 - DNA, 21 bp, synthetic construct
  source 1..21: mol_type = other DNA
  misc_feature 1..21: note = Description of sequence: NOD/SCID R primer
gttggcccctgctaactttct